SEQUENCE LISTING (15 sequences)

SEQ ID NO: 1 - DNA, 23 bp, Artificial Sequence
  Description of Artificial Sequence PRIMER L
ttccaaggcagaacatttgagac

SEQ ID NO: 2 - DNA, 21 bp, Artificial Sequence
  Description of Artificial SequencePRIMER R
tctgagccagggttgaaaggc

SEQ ID NO: 3 - DNA, 23 bp, Artificial Sequence
  Description of Artificial Sequence mPr
atcttgcttacacagaggacttg

SEQ ID NO: 4 - DNA, 20 bp, Artificial Sequence
  Description of Artificial Sequence mPl
atcgccatcacaatcactgt

SEQ ID NO: 5 - DNA, 25 bp, Artificial Sequence
  Description of Artificial Sequence mPp
atcagagtctggttatgtggcatcc

SEQ ID NO: 6 - DNA, 20 bp, Artificial Sequence
  Description of Artificial Sequence mGAPDH-r
ggcatcgaaggtggaagagt

SEQ ID NO: 7 - DNA, 20 bp, Artificial Sequence
  Description of Artificial Sequence m-GAPDH-l
atggccttccgtgttcctac

SEQ ID NO: 8 - DNA, 26 bp, Artificial Sequence
  Description of Artificial Sequence mGAPDH-p
gaaggtggtgaagcaggcatctgagg

SEQ ID NO: 9 - protein, 22 aa, Artificial Sequence
  Description of Artificial Sequence SYNTHETIC PEPTIDE CORRESPONDING TO MOUSE UTEROGLOBIN AMINO ACID SEQUENCE SPANNING RESIDUES LYS28-THR49
KPFNPGSDLQNAGTQLKRLVDT

SEQ ID NO: 10 - DNA, 19 bp, Artificial Sequence
  Description of Artificial Sequence hUGr
tacacagtgagctttgggc

SEQ ID NO: 11 - DNA, 19 bp, Artificial Sequence
  Description of Artificial Sequence hUGI
atgaaactcgctgtcaccc

SEQ ID NO: 12 - DNA, 20 bp, Artificial Sequence
  Description of Artificial Sequence hUGp
tgaagaagctggtggacacc

SEQ ID NO: 13 - DNA, 20 bp, Artificial Sequence
  Description of Artificial Sequence hGAPDH-r
caaagttgtcatggatgacc

SEQ ID NO: 14 - DNA, 18 bp, Artificial Sequence
  Description of Artificial Sequence hGAPDH-I
ccatggagaaggctgggg

SEQ ID NO: 15 - DNA, 20 bp, Artificial Sequence
  Description of Artificial Sequence hGAPDH-p
tcctgcaccaccaactgctt

(19) United States
(12) Patent Application Publication
(10) Pub. No.: US 2002/0160948 A1
(43) Pub. Date: Oct. 31, 2002

(54) RECOMBINANT HUMAN UTEROGLOBIN IN TREATMENT OF INFLAMMATORY AND FIBROTIC CONDITIONS

(76) Inventors: APRILE PILON, GAITHERSBURG, MD (US); ANIL B. MUKHERJEE, BROOKEVILLE, MD (US); ZHONGJIAN ZHANG, ROCKVILLE, MD (US)

Correspondence Address: BARRY EVANS, KRAMER LEVIN NAFTALIS & FRANKEL, 919 THIRD AVENUE, NEW YORK, NY, 10022, US

(21) Appl. No.: 09/120,264

(22) Filed: Jul. 21, 1998

(51) Int. Cl. A61K 38/00; G01N 33/53; C07K 1/00; C07K 14/00; C07K 17/00

(52) U.S. Cl. 514/12; 530/350; 435/7.1; 435/975

(57) ABSTRACT

Compositions and methods for preventing or treating primary cancer cell growth and tumor metastasis, as well as stimulation of hematopoiesis are described and claimed. The present invention also relates to methods of treating cancer and uteroglobin receptor-related conditions by targeting a uteroglobin receptor with recombinant human uteroglobin (rhUG). Also disclosed and claimed are methods of purifying a uteroglobin receptor and methods of using such receptor(s) to identify uteroglobin structural analogs and UG-receptor ligands.

### CROSS-REFERENCE TO RELATED APPLICATIONS

The present application is a continuation-in-part of U.S. Application Serial No. 09/087,210, filed May 28, 1998, which is a continuation-in-part of U.S. application Ser. No. 08/864,357, filed May 28, 1997. The disclosures of each of the aforementioned applications are incorporated herein by reference.

### FIELD OF THE INVENTION

The invention relates generally to the treatment of inflammatory and fibrotic, conditions using native human uteroglobin (hUG) or recombinant human uteroglobin (rhUG). Novel physiological roles and therapies for UG (hUG or rhUG) have been identified. Specifically, the invention relates to the treatment of inflammatory and fibrotic conditions by administering hUG or rhUG to inhibit PLA₂s and/or to prevent fibronectin deposition. The invention further provides a method for the treatment of neonatal respiratory distress syndrome (RDS) and bronchopulmonary dysplasia (BPD), a critical clinical condition of the lung, and glomerular nephropathy, a disease of the kidney, both characterized by the inflammatory and fibrotic conditions. The invention also provides methods for the treatment of cancer by administering uteroglobin to mediate tumor suppression via its receptor. Further, the invention provides methods of purifying the uteroglobin receptor(s) from cells producing such receptors and using such purified receptors to identify UG-receptor ligands and uteroglobin structural analogs.

Documents cited in this application relate to the state-of-the-art to which this invention pertains, each of which is incorporated herein by reference.

### BACKGROUND OF THE INVENTION

Inflammatory, Fibrotic and Cancerous Conditions

The search for improved therapeutic agents for the treatment of inflammatory and fibrotic diseases has received much attention in recent years. Neonatal RDS, a lung surfactant deficiency disease, is a condition of particular interest in that it is one of the major causes of mortality in premature neonates. While introduction of surfactant therapy dramatically improves survival of RDS patients, the development of chronic inflammatory and fibrotic disease in a significant percentage of this patient population is a major problem. Likewise, hereditary fibronectin-deposit glomerular nephropathy leads to end stage renal failure when patients' kidneys become blocked and no longer filter the blood. Nephropathy is characterized by fibronectin deposits and fibrosis of the kidneys which render the organ non-functional, and eventually, unable to support life.

PLA₂ (phospholipase A2), a class of endogenous enzymes that hydrolyze the Sn2 position ester bond of glycerophospholipids, is one of many proteins implicated in inflammatory and fibrotic conditions. It is also responsible for hydrolysis of surfactant phospholipids in the lungs. UG (also known as CC10, CC16, CC17, urine protein-1, P-1, progesterone binding protein, PCB-binding protein, Clara cell secretory protein (CCSP), blastokinin, retinol-binding protein, phospholipid-binding protein, and alpha2-microglobulin) inhibits the activity of PLA₂ in vitro.

Uteroglobin is a small globular homodimeric protein. It has a molecular weight of 15.8 kDa, but it migrates in electrophoretic gels at a size corresponding to 10 kDa. Human uteroglobin is abundant in the adult human lung, and comprises up to about 7% of the total soluble protein. However, its expression is not fully activated in the developing human fetus until late in gestation. Consequently, the extracellular lung fluids of pre-term infants contain far less human UG than those of adults. UG is also expressed by the pancreas.

PLA₂s play critical roles in the inflammatory response because they release arachidonic acid (AA) from cellular phospholipid reservoirs. AA is metabolized to a number of potent inflammatory mediators in a process referred to as the arachidonic acid cascade.

Several acute and chronic clinical conditions have been characterized by elevated serum or local PLA₂ activity (see Table 1, below). Table 1. Clinical Conditions Associated with PLA₂ Activity

TABLE 1
Clinical Conditions Associated with PLA₂ Activity

| Diseases | Sites |
|---|---|
| Rheumatoid arthritis | Serum, synovial fluid, WBC |
| Collagen vascular diseases | Serum |
| Pancreatitis | Serum |
| Peritonitis | Peritoneal fluid and cells |
| Septic shock | Serum |
| ARDS^{a} | Serum and alveolar fluid |
| Acute renal failure | Serum |
| Autoimmune uveitis | Serum, aqueous humor |
| Bronchial asthma | Bronchial fluid |

^{a}Adult respiratory distress syndrome

There are no effective PLA₂ inhibitors presently available for clinical use. To date, only a few PLA₂ inhibitors have progressed into clinical trials, but none have qualified for commercial marketing.

Fibronectin (Fn) is a 200 kDa glycoprotein which exists in several different forms and is secreted by different tissues. Fn is an essential protein and targeted disruption of the Fn gene in mice showed that it has a central role in embryogenesis. Fn also plays a key role in inflammation, cell adhesion, tissue repair and fibrosis, and is deposited at the site of injury. Plasma fibronectin (pFn) is secreted by the liver and circulates in the plasma. In the lung, cellular Fn (cFn) is secreted upon inflammation and injury. Both types of Fn are chemotactic factors for inflammatory cells and fibroblasts. Large numbers of inflammatory cells and fibroblasts infiltrate the lung during inflammatory episodes, which can lead to pulmonary fibrosis and ultimately death. Elevated levels of Fn have been detected in human clinical conditions such as neonatal RDS and BPD of the lung, and glomerular nephropathy of the kidney.

The search for improved cancer therapeutic and prophylactic agents represents an ongoing challenge for science and medicine. Because cancer cells are not recognized by the immune system as foreign, they are able to grow unchecked until vital bodily functions are affected. Current therapeutic regimens consist primarily of chemotherapeutic agents and irradiation, both of which are highly toxic to normal cells as well as to tumor cells. To date, no naturally occurring, non-toxic, extracellular suppressors of tumor cell growth have been found.

The Role of UG

Amino acid analysis of purified human UG reveals that it is structurally similar but not identical to other “UG-like” proteins, e.g. rabbit UG. 39 of 70 amino acids are identical between human and rabbit UG (see FIG. 1). The “UG-like” proteins, including human UG/CC10, rat CC10, mouse CC10, and rabbit UG, exhibit species-specific and tissue-specific antigenic differences, as well as differences in their tissue distribution and biochemical activities in vitro. UG-like proteins have been described in many different contexts with regard to tissue and species of origin, including rat lung, human urine, sputum, blood components, rabbit uterus, rat and human prostate, and human lung. At present there are no known physiological roles for these proteins.

Despite years of study, the biological roles of these proteins in vivo remain unclear. The absence of structural identity among UG-like proteins makes it impossible to predict whether a protein will possess in vivo therapeutic function in humans based on in vitro or other activity exhibited by a structurally related protein. For example, human uteroglobin binds less than 5% of the amount of progesterone as rabbit UG binds in the same assay. Human UG has a lower isoelectric point (4.6) than rabbit UG (5.4).

Stripp et al. (1996) have reported studies on a uteroglobin knockout mouse generated to eliminate expression of uteroglobin. The mouse has Clara cells which exhibit odd intracellular structures in place of uteroglobin secretion granules, but there is no other phenotype. This observation is highly significant because pulmonary function accompanied by pulmonary inflammation and fibrosis was expected. Moreover, this knockout mouse showed no evidence of renal, pancreatic, or reproductive abnormality, indicating that the uteroglobin protein had no significant role in controlling inflammation of fibrosis in vivo.

Leyton et al. (1994) reported the anti-metastatic properties of uteroglobin which were attributed to its inhibition of the release of arachidonic acid by tumor cells. (U.S. Pat. No. 5,696,092 to Patierno et al.). Kundu et al. (1996) continued this work with the observation of inhibition of ECM invasiveness by a variety of tumor cell types. ECM invasion correlated with the presence of a 190 kDa uteroglobin binding protein in responsive cell types. The ECM invasion activity of cells lacking this protein could not be inhibited by uteroglobin.

### OBJECTS OF THE INVENTION

Therefore, it is an object of the present invention to provide a method of preventing or treating primary cancer cell growth including administering a tumor-suppressive effective amount of recombinant human uteroglobin (rhUG) or a fragment or derivative thereof.

It is a further object of the invention to provide a pharmaceutical composition consisting of a tumor-suppressive effective amount of rhUG and a pharmaceutically acceptable carrier or diluent. Such compositions should consist of a mixture of reduced and non-reduced, monomeric and dimeric rhUG, and preferably, the composition should consist of reduced monomeric rhUG.

It is an additional object of the invention to provide a method of preventing or treating tumor metastasis by inhibiting fibronectin aggregation and/or deposition including administering a fibronectin inhibiting effective amount of rhUG or a fragment or derivative thereof.

Further, it is an object of the invention to provide a method of stimulating hematopoiesis consisting of administering a hematopoiesis stimulating effective amount of rhUG or a fragment or derivative thereof.

Still further, it is an object of the invention to provide a pharmaceutical composition comprising a hematopoiesis stimulating effective amount of rhUG or a fragment or derivative thereof and a pharmaceutically acceptable carrier or diluent.

It is an additional object of the invention to provide a method of purifying a uteroglobin receptor(s) from a sample, wherein the method includes the following steps:

(a) contacting said sample with rhUG bound to a solid support; and

(b) eluting a purified sample of uteroglobin receptor(s) from said solid support.

Further, it is an object of the invention to provide purified uteroglobin receptor(s) for use in screening samples containing compounds, peptides or proteins which are uteroglobin structural analogs and/or UG-receptor ligands.

Finally, it is an object of the invention to provide methods of targeting a uteroglobin receptor(s) by administration of an effective amount of rhUG for the treatment or prevention of primary cancer cell growth and tumor metastasis, and for stimulating hematopoiesis.

### SUMMARY OF THE INVENTION

It has now been found that uteroglobin plays a central physiological role in inhibition of PLA₂s and in prevention of fibronectin deposition and fibrosis in vivo. A combination of experiments performed in a new strain of transgenic uteroglobin “knockout” mice, and in a monkey model of neonatal respiratory distress syndrome (RDS) which involves pulmonary inflammation and fibrosis demonstrate these effects. The uteroglobin knockout mice of the present invention (hereinafter the “UG KO mice/mouse”) exhibit lethal glomerular nephropathy and renal parenchymal fibrosis, as early and late onset diseases, respectively. Administration of exogenous Fn to normal mice causes Fn deposition in the kidneys, but administration of equimolar amounts of Fn and rhUG does not.

Reduction of PLA₂ activity in vivo has been demonstrated in the presence of rhUG. In a first experiment, the phenotype of the UG KO mice revealed that serum PLA₂ activity is significantly elevated in the absence of UG, compared to PLA₂ activity in littermates possessing a functional UG gene. In a second experiment, administration of rhUG to pre-term monkeys suffering from RDS was shown to inhibit PLA₂ activity in the extracellular fluids of the lungs.

Other experiments demonstrate that in vitro PLA₂ can degrade the artificial surfactant (typically Survanta) used in treatment of RDS and that UG can inhibit this degradation. These experiments demonstrate that UG mediates PLA₂ inhibition and Fn deposition in vivo following intratracheal or intravenous administration.

Experiments with the uteroglobin knockout mouse demonstrate that rhUG may be used to treat conditions in which uteroglobin is found to be deficient or the protein itself bears a loss-of-function mutation. It has now been discovered that rhUG may be used to treat or prevent inflammatory or fibrotic conditions in which functional endogenous uteroglobin is deficient in the circulation or at the site of inflammation or fibrosis. Reductions in the levels of hUG in serum and/or broncho-alveolar lavage fluids have been found in certain pulmonary inflammatory or fibrotic conditions, including pre-term infants at risk for developing neonatal BPD. It has been found that UG may be used to supplement deficient or defective endogenous uteroglobin to prevent or treat such inflammatory and fibrotic conditions.

In adenocarcinomas and in oncogenic virus-transformed epithelial cells, the uteroglobinexpression is either drastically reduced or totally absent. By stably-transfecting adenocarcinoma cells lines with a human UG (hUG)-cDNA construct, forced UG-expression has been found to suppress anchorage-independent growth and extracellular matrix-invasion by only those cells that express the UG-receptor(s). Treatment of these receptor-positive cells with purified hUG yielded identical results. These data define both autocrine and paracrine pathways by which UG exerts its suppressive effects on cancer cells. This is the first demonstration of an extracellular tumor suppressor and the first indication that uteroglobin can be used to treat primary tumors and their metastasis. Further, aged UG deficient mice have now been found to develop tumors, confirming the tumor suppressor properties of UG in vivo.

According to one aspect, the invention provides a method of preventing or treating primary cancer cell growth consisting of administering a tumor-suppressive effective amount of recombinant human uteroglobin (rhUG) or a fragment or derivative thereof.

According to a further aspect, the invention provides a method of preventing or treating primary cancer cell growth consisting of targeting a uteroglobin receptor by administering a tumor-suppressive effective amount of recombinant human uteroglobin (rhUG) or a fragment or derivative thereof.

In accordance with a further aspect, the invention provides a pharmaceutical composition consisting of a tumor-suppressive effective amount of rhUG and a pharmaceutically acceptable carrier or diluent. In a preferred embodiment, rhUG is reduced and monomeric and has a purity of about 75% to about 100%, preferably about 90% to 100%, and most preferably at least about 95%.

A further aspect of the invention provides a method of preventing or treating metastasis by inhibiting fibronectin aggregation and/or deposition consisting of administering a fibronectin inhibiting effective amount of rhUG or a fragment or derivative thereof. This aspect of the invention also includes targeting a uteroglobin receptor by administering a fibronectin inhibiting effective amount of rhUG.

In accordance with a further aspect, the invention provides a method of stimulating hematopoiesis consisting of administering a hematopoiesis stimulating effective amount of rhUG or a fragment or derivative thereof, wherein the method may also include targeting a uteroglobin receptor by administration of rhUG.

The invention also provides a pharmaceutical composition consisting of a hematopoiesis stimulating effective amount of rhUG or a fragment or derivative thereof and a pharmaceutically acceptable carrier or diluent, wherein the rhUG has a purity of about 75% to about 100%, preferably about 90% to 100%, and most preferably at least about 95%.

In another aspect of the invention, a method of purifying a uteroglobin receptor(s) from a sample of cells producing such receptor(s) is provided, consisting of contacting a sample with rhUG bound to a solid support, followed by eluting a purified sample of uteroglobin receptor(s) from said solid support.

The invention also includes a method of preparing reduced rhUG consisting of contacting oxidized rhUG with a reducing agent, e.g., dithiothreitol or B-mercaptoethanol, for a time and temperature sufficient to reduce rhUG. In a preferred embodiment, the reduced rhUG is monomeric.

Further, the present invention provides a method of generating antibodies to a uteroglobin receptor consisting of immunizing an animal with a purified uteroglobin receptor(s) and isolating antibodies directed against a uteroglobin receptor.

Finally, the invention provides uteroglobin receptor(s) as a means to screen samples for compounds, peptides and proteins which are uteroglobin structural analogs and UG-receptor ligands. In this regard, uteroglobin receptor(s) may be used in a kit for screening for such compounds, peptides or proteins for those which are uteroglobin structural analogs and/or UG-receptor ligands.

### BRIEF DESCRIPTION OF THE DRAWINGS

The invention will now be described in more detail, with reference to the accompanying drawings, in which:

FIG. 1 shows an alignment of UG-like proteins;

FIG. 2A shows the intended targeting construct of the transgenic UG knockout mouse; the restriction sites are B—BamIII, E=EcoRI, H=HindIII;

FIGS. 2B-2D show verification of the genetic construct in progeny of transgenic embryos by PCR and Southern blot analyses; (B) southern blot analyses of the targeted ES R1 cell clones, wherein Wt=wild type; (C) representative PCR analyses of genomic DNA from tail biopsies of offspring; the genotypes and their corresponding PCR products are as follows: UG^{+/+},304 bp; UG^{+/-},304 and 667 bp; UG^{-/-},667 bp; (D) southern blot of mouse tail genomic DNA;

FIG. 2E shows confirmation of the absence of UG-mRNA in the lung tissues of UG^{-/-} mice by RT-PCR analysis; RT-PCR analyses of total RNA extracted from the lung tissues of littermates with UG^{+/+}, UG^{+/-}, and UG^{-/-} genotypes; a 273 bp RT-PCR product was detectable in the lungs of UG^{+/+}, and UG^{+/-}, but lacking from those of UG^{-/-} mice;

FIG. 2F shows confirmation of the absence of UG protein in the lungs of UG^{-/-} mice by Western analysis; proteins (30 micrograms each) from lung lysate were resolved by electrophoresis using 4-20% gradient SDS-Page under non-reducing conditions and immunoblotted using rabbit anti-mouse UG;

FIG. 2G shows confirmation of the absence of UG in lung tissue sections of the UG^{-/-}0 mice using immunohistochemical methods in bronchiolar epithelial cells; the dark staining over the bronchiolar epithelial cells of UG^{+/+} mouse (upper panel) indicates UG immunoreactivity; note the absence of immunoreactivity in UG^{-/-} mouse lungs (lower panel).

FIGS. 3A-3J compare histopathological analyses of kidney sections from normal versus UG^{-/-} mice, showing abnormal parenchymal fibrosis and glomerular Fn deposition in the knockout mice only; H & E staining of kidney sections from a UG^{+/+} (A) and its UG^{-/-} (B) littermate; (C) kidney section of a 10 month old mouse with severe parenchymal fibrosis; (D) a region of the same mouse kidney in (C) showing renal tubular hyperplasia (magnification 40×, g=glomerulus; f=fibrosis; t=tubule); (E) transmission electron microscopy of the glomerular deposit of a UG^{-/-} mouse with severe renal disease (magnification 6000×); (F) the inset in (E) is magnified 60,000×, which shows the long striated fibrillar structures indicative of collagen (col) and short diffuse ones consistent with Fn fibrils; (G) Fn immunofluorescence of a kidney section from a UG^{+/+} mouse using murine Fn-antibody; (H) Fn-immunofluorescence of a kidney section from a UG^{-/-} mouse with severe renal disease; Mason's trichrome staining of the kidney sections with UG^{+/+} (I) and UG^{-/-} (J) mice; the bluish staining over the glomeruli of UG^{-/-} mouse kidney section is collagen (magnification approximately 40×).

FIG. 4A shows the presence of Fn aggregates only in the kidneys of the UG^{-/-} mice; immunoprecipitation and western blotting of Fn from plasma, kidney, and liver of UG^{+/+} and UG^{-/-} mice; a multimeric FN band (bold arrow) was detected only in the kidney lysates of UG^{-/-} mice.

FIGS. 4B and 4C show the formation of UG-Fn complexes in vitro; (B) equimolar concentrations of UG and Fn were incubated, immunoprecipitated with and detected by Western blotting with either Fn or UG antibody; the immunoprecipitates contain both Fn (lane 2, upper panel) and UG (lane 2, lower panel); lanes 1 of both panels represent Fn and UG standards; (C) equimolar concentrations of ¹²⁵I-UG and Fn were incubated at 4C for 1 hour and the resulting complex was resolved by electrophoresis on 6% non-reducing, non-denaturing polyacrylamide gels; lane 1, coomassie blue stained Fn-UG heteromer; lane 2, its autoradiogram.

FIG. 4D shows the presence of UG-Fn complexes in the plasma of normal but not UG^{-/-} mice; immunoprecipitation of plasma from UG^{+/+} and UG^{-/-} mice with Fn-antibody and western blotting with Fn and UG antibodies; Fn (upper panel); UG (lower panel); std=standards for UG and Fn.

FIG. 4E shows the dose-dependent inhibition of Fn self-aggregation by UG in vitro; affinity-crosslinking of ¹²⁵I-Fn with unlabeled Fn in the absence (lane 2) and presence of varying amounts of UG (lanes 3-5); the intensity of the very high molecular weight, radioactive Fn band (land 2) formed in the absence of UG is reduced in a dose-dependent manner; lane 1, ¹²⁵I-Fn with unlabeled Fn in the absence of UG and DSS; open arrowhead−multimeric Fn; lower thin arrow=220 kDa Fn.

FIG. 4F shows the inhibition of Fn-collagen complex formation by UG; affinity crosslinking of ¹²⁵I-collagen I with unlabeled Fn in the absence of (lane 3) and presence (lane 4) of UG; lane 1, coomassie blue-stained collagen I; alpha₁-alpha₁ chain of collagen I and alpha₂-alpha₂ chain of collagen I; lane 2, ¹²⁵I-collagen I and unlabeled Fn in the absence of UG and DSS.

FIGS. 5A-5F show the immunohistochemical analysis of Fn deposition in the kidneys of normal and UG^{-/-} mice only in the absence of UG; (A) kidney section of a wild-type mouse that received a mixture of equimolar concentrations of Fn and UG intravenously; (B) UG^{+/+} mouse that received the same dose of Fn as in (A) but without UG; (C) apparently healthy, UG^{-/-} mouse receiving a mixture of Fn and UG; (D) UG^{-/-} mouse receiving Fn alone (same dose as in (C), but without UG; (E) Fn-fibrillogenesis by cultured cells grown in medium supplemented with soluble hFn alone; (F) a cell culture identical to one (E) which was fed with medium containing a mixture of equimolar concentrations of soluble hFn and UG (magnification 40×, g glomerulus).

FIGS. 6A-6B show the format for a diagnostic assay to detect UG-Fn complexes in clinical samples.

FIG. 7 shows the passage of UG dimer through an 8.0 kDa MWCO dialysis membrane but nqot a 3.4 kDa MWCO dialysis membrane.

FIG. 8 shows a Scatchard plot of specific binding of ¹²⁵-I hUG (reduced) on NIH 3T3 cells. The data are from three experiments and each data point represents the mean of triplicate determinations.

FIG. 9 shows an autoradiograph of an SDS-Page analysis of the affinity crosslinking of hUG-binding proteins on NIH 3T3 (lanes 1-3), mastocytoma (Lanes 4-5), sarcoma (lanes 6-7) and lymphoma (lanes 8-9) cells. Reduced ¹²⁵I-hUG was incubated with each of these cells in the absence or presence of unlabeled reduced hUG for binding and then crosslinked with disuccinimidyl suberate (DSS) (lane 1: (−) DSS; lane 2: (+) DSS; lane 3: (+) unlabeled hUG, (+) DSS; lane 4: (+) DSS; lane 5: (+) unlabeled hUG, (+) DSS; lane 6: (+) DSS; lane 7: (+) unlabeled reduced hUG, (+) DSS; lane 8: (+) DSS; and lane 9: (+) unlabeled reduced hUG, (+) DSS).

FIG. 10 shows an autoradiograph of an SDS-Page analysis of affinity purified UG-binding protein(s).

FIG. 11 shows an autoradiograph of an SDS-Page analysis of the effect of different cytokines and other agents on the expression of UG-binding proteins by NIH 3T3 cells.

FIG. 12A shows RT-PCR analysis of total RNA extracted from pRC/RSV-hUG-transfected and wild type (WT) adenocarcinomas of the uterus and prostate. Lanes 1 and 2 represent different clonal isolates.

FIG. 12B shows Western blot analysis of uteroglobin proteins produced by the non-transfected and transfected cell lines.

FIGS. 13A and 13B shows the effect of induced-expression of hUG on ECM invasion by HEC-LA cells.

FIG. 14 shows the morphology of the control cells (pRC/RSV vector alone transfected adenocarcinomas of the uterus) on soft agar was shown in (a) HEC-1A, while morphology of the hUG expression construct transfected cells on soft agar was shown in (b) HEC-1A/UG.

FIG. 15 shows the presence of the UG-receptor on HEC-LA (responder) cells but not on HTB-81 (non-responder) cells (Lane 1: (−) DSS; lane 2: (+) DSS; and lane 3: (+) hUG, (+) DSS). Affinity crosslinking of ¹²⁵I-hUG with its binding proteins on non-transfected (a) and pRSV/hUG-transfected HEC-1A and HTB-81 cells, respectively, are shown. The cells were incubated with reduced ¹²⁵I-hUG in the absence and presence of unlabeled reduced hUG for binding and then crosslinked with DSS.

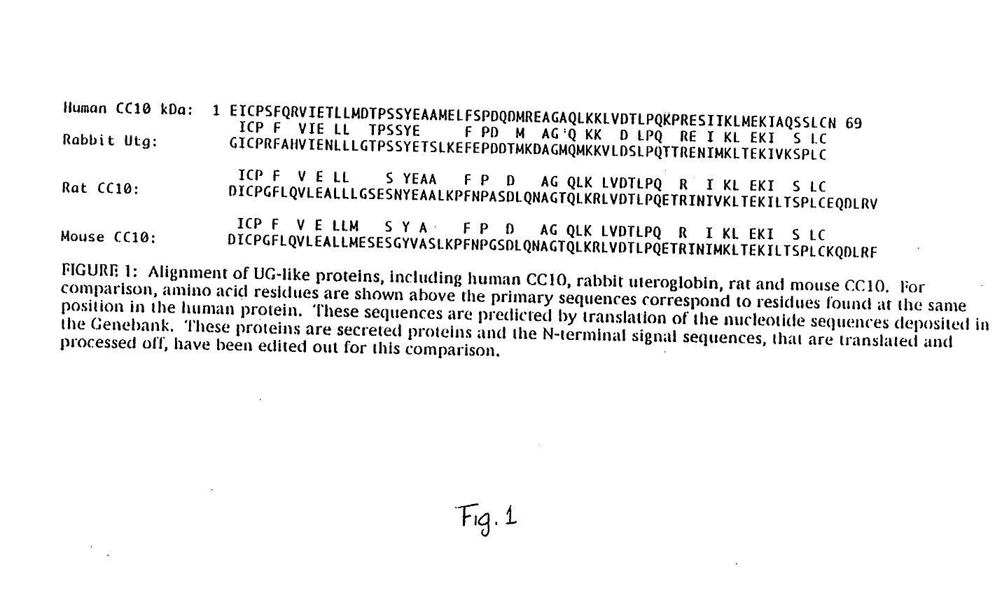

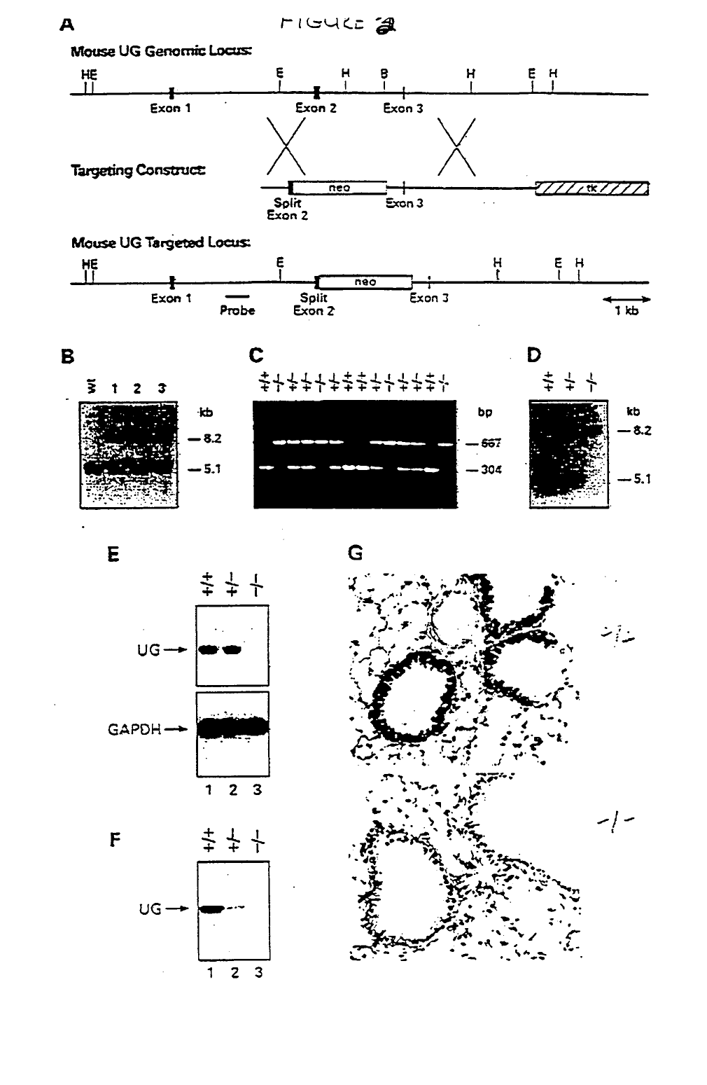

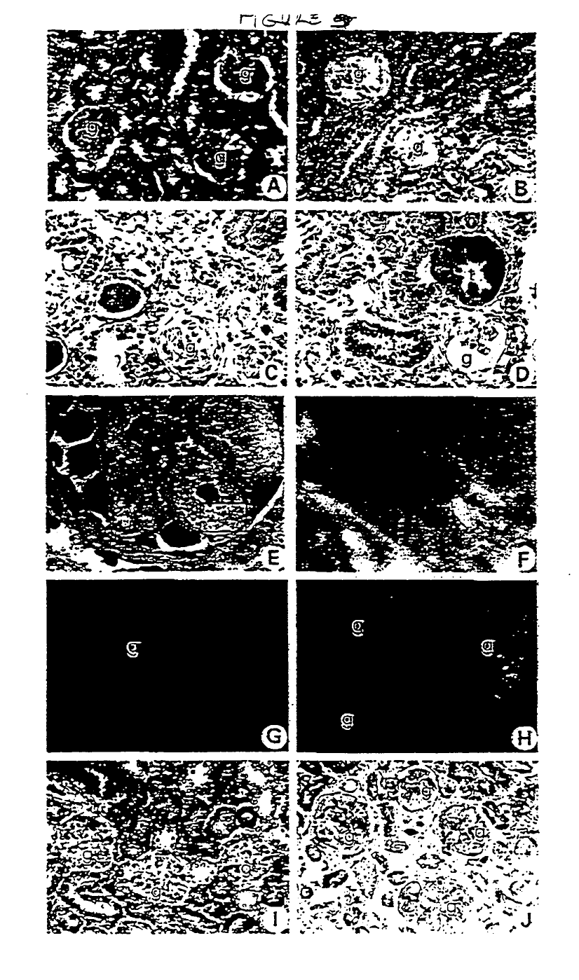

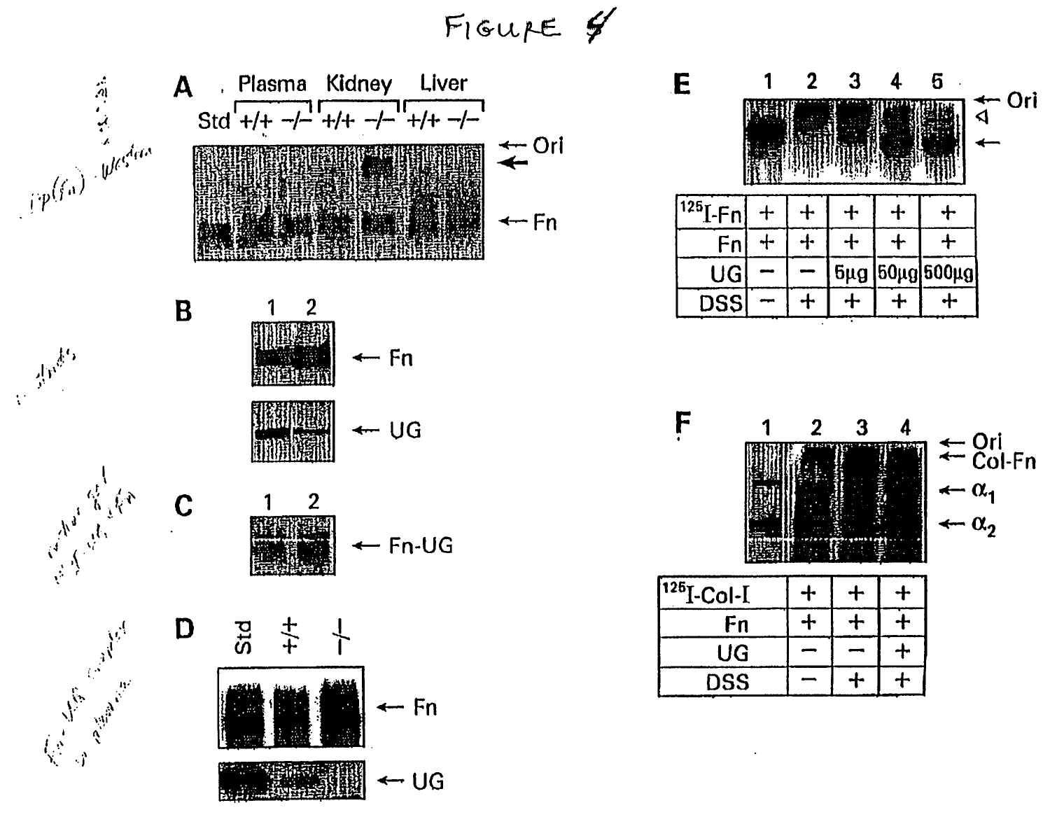

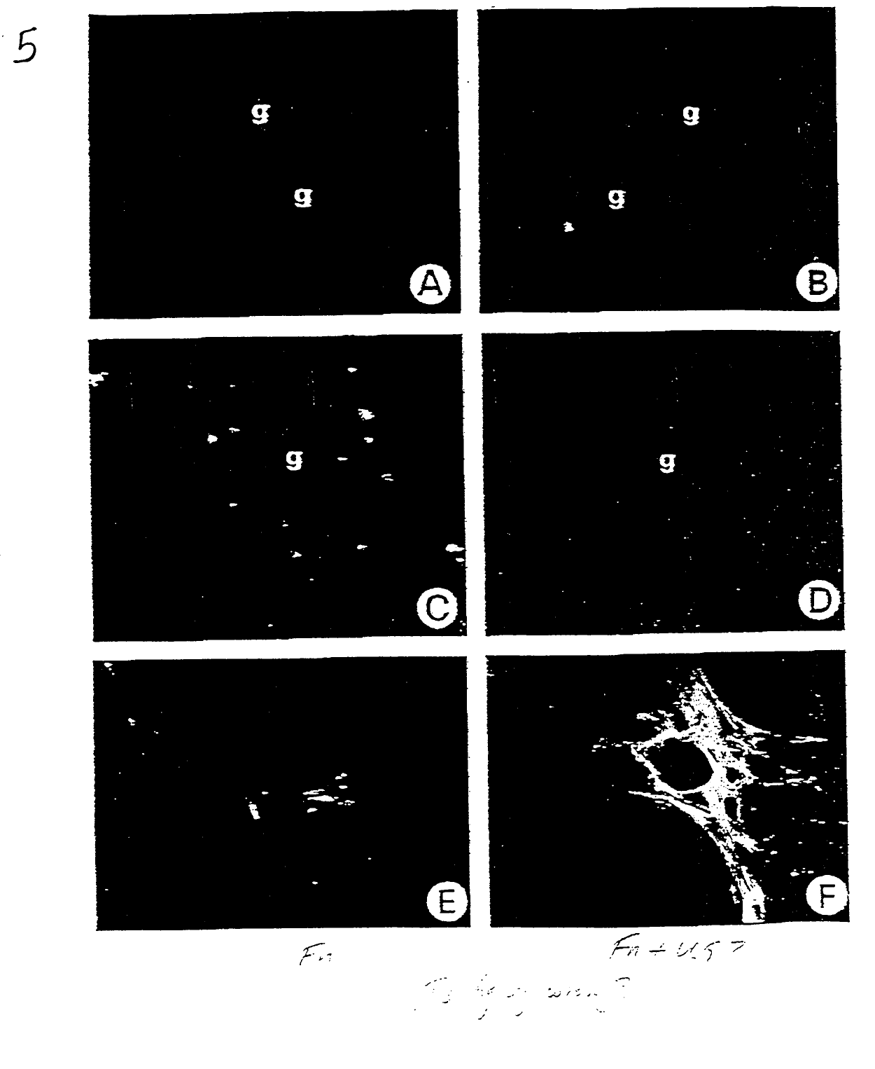

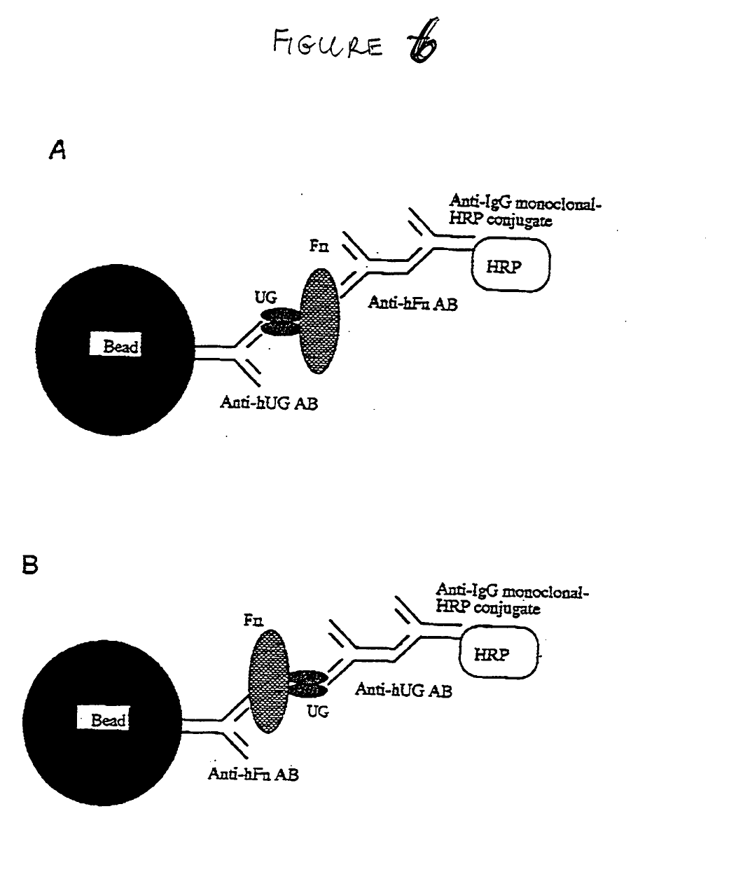

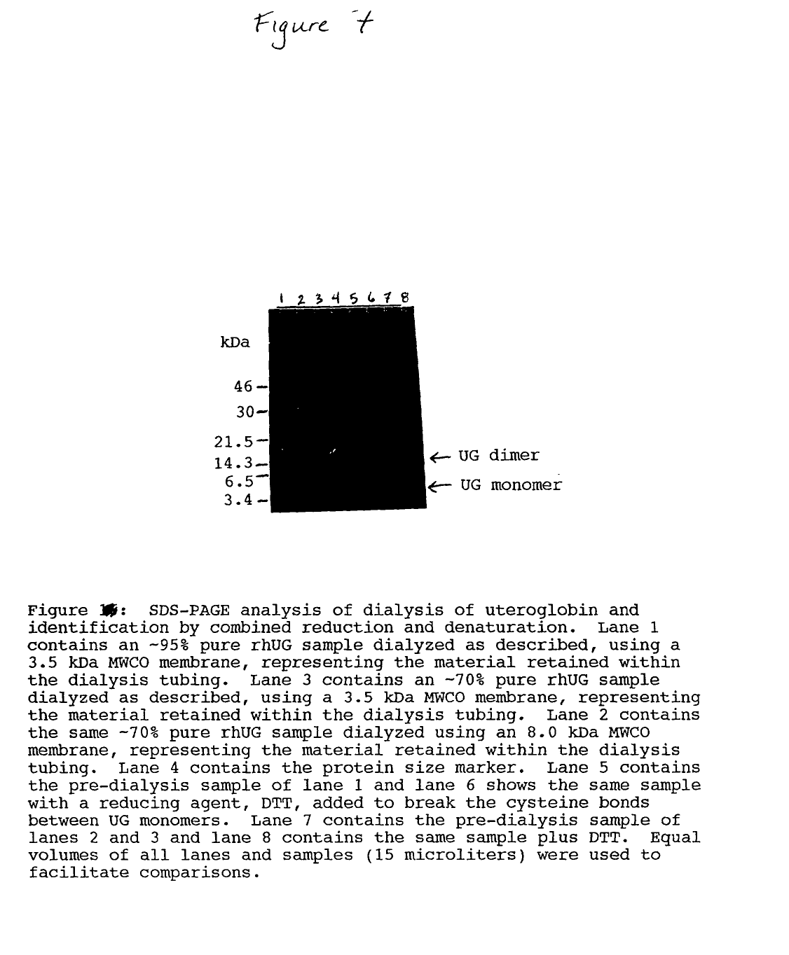

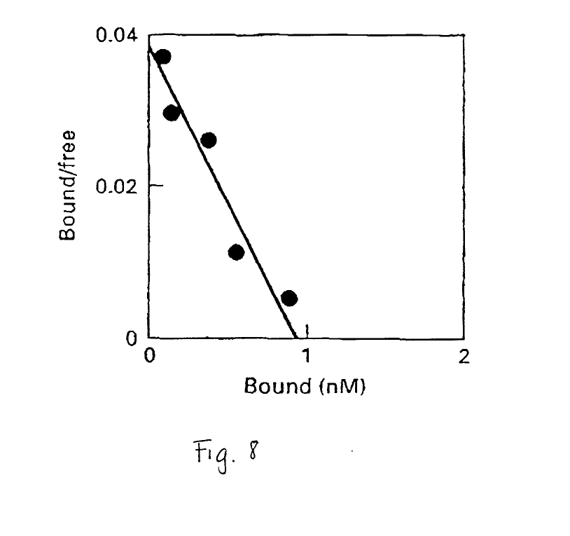

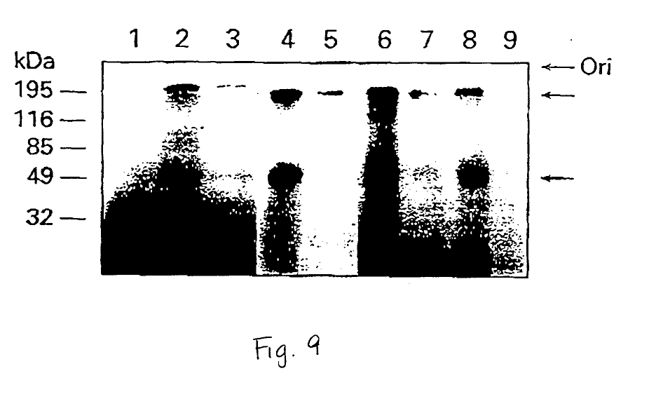

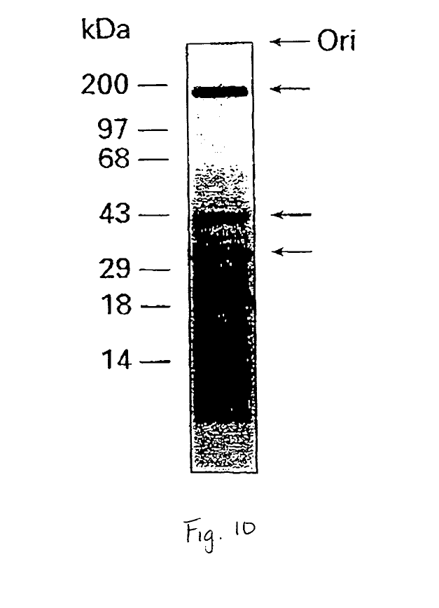

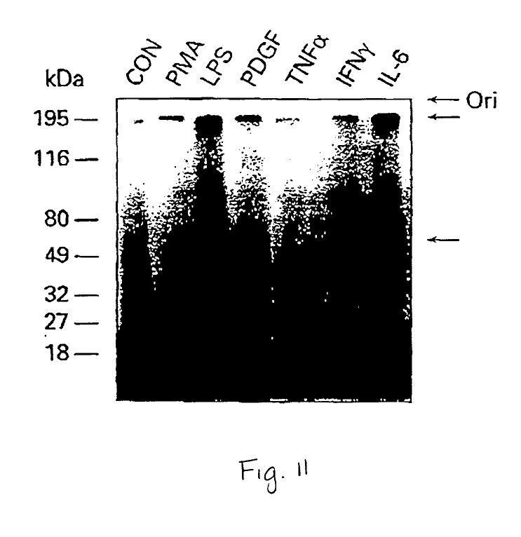

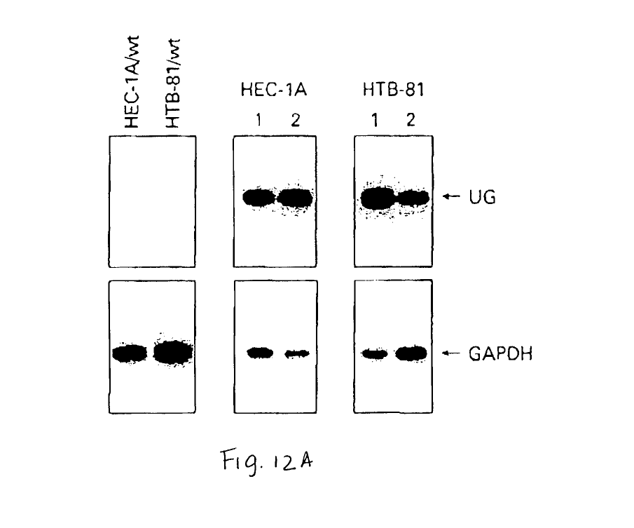

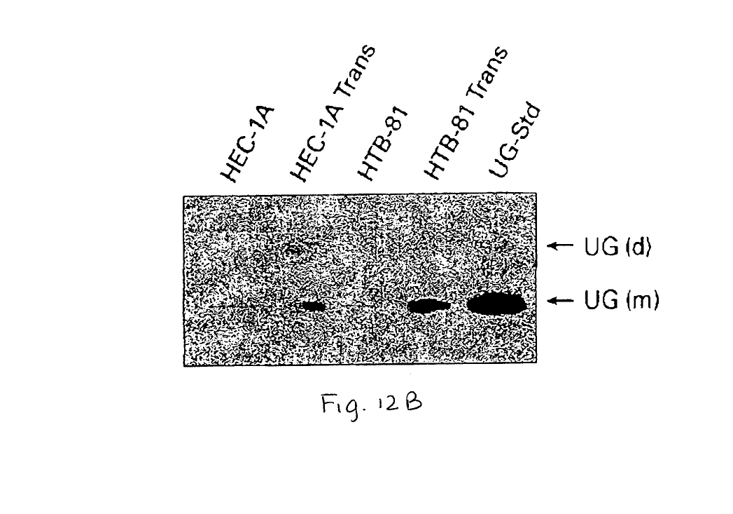

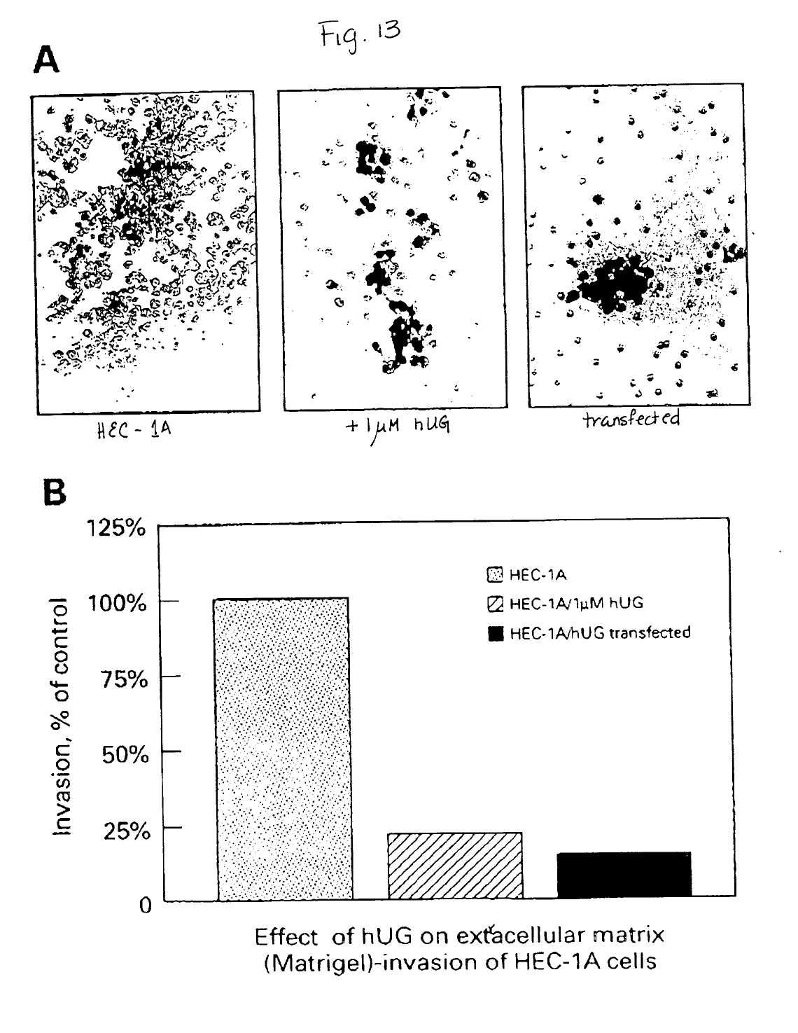

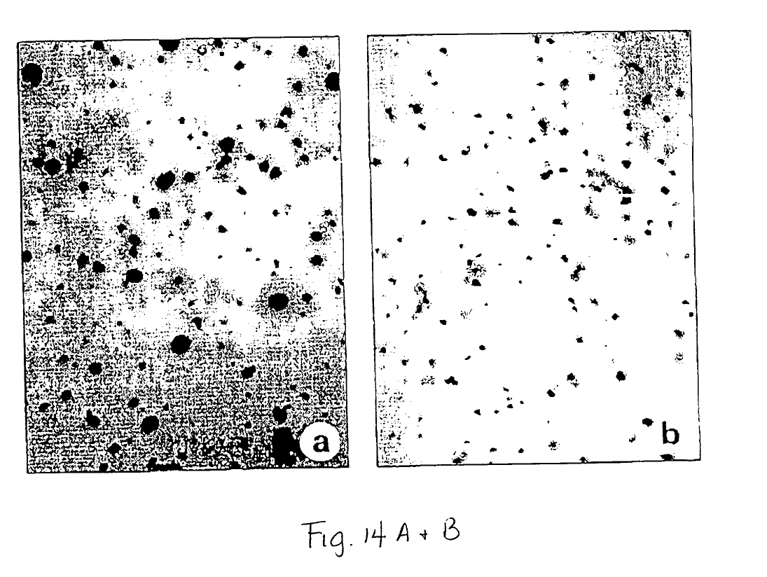

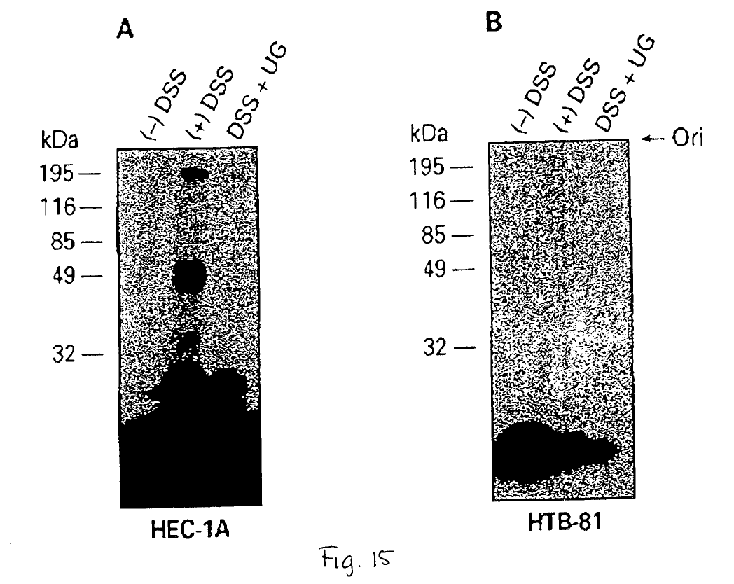

### DETAILED DESCRIPTION OF PREFERRED EMBODIMENTS

rhUG

The rhUG of the invention has substantially the same amino acid sequence as that of the native human UG protein. An amino acid sequence having “substantially the same” amino acid sequence as that of the native human protein includes rhUG having at least 75% identity to the native human protein. In a preferred embodiment, rhUG has at least 85% identity, and in a most preferred embodiment, rhUG has at least 98% identity to the native UG.

Also included in the method of the present invention is the use of fragments or derivatives of UG. A “fragment” of UG refers to a portion of the native hUG amino acid sequence having six or more contiguous amino acids of the native protein sequence. The term “derivative” refers to peptide analogs of UG, including one or more amino acid substitutions and/or the addition of one or more chemical moieties, e.g., acylating agents, sulfonating agents, carboxymethylation of the disulphide bonds, or complexed or chelated metal or salt ions, e.g. Mg⁺², CA⁺² or Na⁺¹, with the proviso that the derivative retains the biological activity of the parent molecule.

A “UG-like” protein includes those isolated from mouse, rat, rabbit, etc. having substantially the same amino acid sequences and/or substantial sequence similarity with native human uteroglobin. With regard to sequence similarity, like-amino acids may be substituted in a UG-like protein, e.g. tyrosine for phenylalanine or glycine for alanine. UG-like proteins which are considered substantially similar have approximately 30% sequence similarity, preferably 50% sequence similarity, more preferably at least 75% sequence similarity, and most preferably at least 90-95% sequence similarity. UG-receptor ligands are peptide, protein or chemical moieties (e.g. organic ligands) that bind to the UG receptor and mediate all or part of its activities. Uteroglobin structural analogs are compounds, peptides or proteins, or fragments or derivatives thereof having substantially similar secondary and tertiary structural characteristics when compared to native uteroglobin, such that a structural analog retains at least 50% and preferably at least 75% of the activity of native protein. In a most preferred embodiment, a structural analog retains at least 90% of the activity of the native protein.

Further, the UG used in the method of the present invention is substantially pure. The term “substantially pure” refers to UG having a purity of about 75% to about 100%. In a preferred embodiment, UG has a purity of about 90% to about 100%, and in the most preferred embodiment, UG has a purity of at least 95%.

Clinical Uses of UG

The invention provides, in another aspect, a method of treating or preventing an inflammatory or fibrotic or cancerous condition comprising administering to a mammal, which may be animal or human, an effective amount of UG.

The following non-limiting list of conditions are representative ‘examples of those associated with UG deficiencies, excessive PLA₂ activity, and fibronectin deposition.

TABLE 2
Clinical Uses of Recombinant Uteroglobin
(Grouped by UG Property)

| UG Property | Condition(s) |
|---|---|
| UG deficiency | (1) Neonatal broncho-pulmonary dysplasia; |
| | (2) Complications of hemodialysis; |
| | (3) Bleomycin lung; |
| | (4) Chronic obstructive pulmonary disease; |
| | (5) Emphysema |
| | (6) Cancer |
| Excessive PLA₂ activity | (1) Systemic inflammation; |
| (inflammation) | (2) Asthma; |
| | (3) Cystic fibrosis; |
| | (4) Ocular inflammation, including Autoimmune uveitis and |
| | corneal transplant surgery; |
| | (5) Conditions associated with obstetrics and gynecology, |
| | including premature labor and fertility (ex vivo) |
| Excessive PLA₂ activity | (1) Autoimmune diseases, including rheumatoid arthritis, |
| (Immune modulation) | Type I diabetes, Inflammatory bowel disease, and |
| | Crohn's disease; |
| | (2) Transplanted organ rejection |
| Fibronectin deposition | (1) Renal fibroses |
| | (2) Pulmonary fibroses, including idiopathic pulmonary |
| | fibrosis; |
| | (3) Vascular fibrosis |
| | (4) Cancer |
| Binding to cellular receptors | (1) Cancer |
| | (2) Autoimmune diseases |
| | (3) HIV infection |
| | (4) White and red blood cell deficiencies |

Typical relationships between UG deficiency, PLA₂ activity, fibronectin aggregation and deposition in human inflammatory/fibrotic conditions, tumor suppression and uteroglobin receptor(s) are summarized below.

Neonatal BronchoPulmonary Dysplasia (Neonatal BPD)

Neonatal BPD is characterized by severe inflammation and irreversible fibrosis of lung tissue in newborn infants, usually as a result of respiratory distress syndrome (RDS). However, this condition may also be caused by meconium aspiration syndrome or infection.

A deficiency of hUG has been implicated in this condition because the synthesis of pulmonary hUG may be coregulated with surfactant, which starts late in gestation. Thus, severely premature neonates may lack UG as well as surfactant. hUG deficiency may cause increased PLA₂ activity and Fn-related fibrosis, which are associated with the inflammation and fibrosis seen in neonatal BPD. Some infants do not respond to synthetic surfactant, which may be due to excess PLA₂ activity. Thus, UG may be used to treat neonatal BPD.

The preferred route of administration is direct instillation via the endotracheal or the systemic routes.

Multiple Organ Failure (MOF)

Excessive PLA₂ activity has been implicated in MOF due to bacterial sepsis or trauma. This condition is characterized by a systemic inflammatory response, involving rapid, massive tissue damage and loss of organ function in the lungs, kidney, pancreas, intestines, and vasculature. Recent evidence points to the MOF trigger as elevated systemic soluble phospholipase A₂ activity, its direct lysis of tissue cell membranes, and hydrolysis of essential phospholipids, such as lung surfactants. Attempts to inhibit PLA₂ directly in clinical settings have been unsuccessful.

In MOF, the amount of endogenous UG is insufficient to counter the super-activation of PLA₂. Exogenously supplied UG can be used to combat MOF.

Remote organ failure (ROF) involves damage to organs other than the organ primarily affected by trauma or infection. Often remote organ failure involves more than one remote organ, resulting in multiple organ failure. For example, pancreatitis is an inflammation of the pancreas in response to alcohol intake, infection, or trauma, that may result in adult respiratory distress syndrome (ARDS), acute renal failure (ARF), and systemic shock. An episode of inflammatory bowel disease or peritonitis can result in ROF/MOF. ROF/MOF is associated with high levels of circulating, activated PLA₂. The systemic application of hUG could prevent ROF/MOF. The immediate injection of UG in patients with ROF/MOF, could reduce the severity or eliminate the PLA₂ mediated organ failure and shock.

Pancreatitis

All forms of pancreatitis involve elevated Type I soluble PLA₂ activity, both systemic and local. Pancreatitis often results in pulmonary insufficiency or ARDS, characterized by elevated soluble PLA₂ activity in the lungs. Therefore, as an inhibitor of soluble Type I PLA₂s in vivo, UG is an excellent candidate for treatment of two forms of acute pancreatitis, and as a preventative measure of pulmonary insufficiency in all acute forms of pancreatitis.

The preferred route of administration is by the intravenous route.

Inflammatory Bowel Disease

Inflammatory Bowel Disease (IBD), including ulcerative colitis, direticulitis, and Crohn's disease, is characterized by elevated local production and activity of Type II soluble PLA₂. Circulating soluble PLA₂ activity may also be elevated in IBD. IBD causes pulmonary insufficiency or ARDS in severe cases, as a result of elevated PLA₂ activity (which is similar to pancreatitis).

The rationale for the application of exogenous UG in IBD is identical to that of pancreatitis: to downregulate the inflammatory response by inhibiting PLA₂, Fn aggregation and/or deposition and to prevent remote organ involvement (lungs and kidneys).

The preferred route of administration is by the intravenous route in hospitalized patients.

Bacterial Pneumonia

BAL fluids of patients who have survived bacterial pneumonia were shown to have 2-3X higher levels of UG than those who died. Bacterial infection of the lungs may overactivate the endogenous soluble PLA₂. UG may be administered to inhibit or control this effect.

The preferred route of administration is via the intratracheal route if the patient is intubated or intravenous if not.

Complications of Dialysis

The major complication of dialysis is thromboses, i.e., spontaneously formed blood clots. These often plug the vascular access port, impairing treatment, as well as causing ischemic, sometimes life-threatening episodes, in the patient. A second problem with hemodialysis patients is inflammation and/or fibrosis of the proximal vein which returns the dialyzed blood to the patient's main circulation. Fibrosis of the proximal vein is usually detected as an increase in resistance, or pressure, against the return of the dialyzed blood. A third problem is fibrosis and closure of the vascular access site, or fistula. A fourth problem is accelerated atherosclerosis and a fifth is loss of residual renal function, most likely due to Fn deposition.

The possibility that endogenous UG is dialyzed away during the procedure provides an explanation for these problems. The selective removal of endogenous UG leaves circulating Fn free to aggregate, forming the foci for blood clot formation or to deposit on red blood cells, priming them for a clotting response by sticking to each other or to the vascular lumen. Transglutaminases (TGs) are enzymes responsible for building macromolecular lattices found in basement membranes, skin and blood clots. In the absence of free UG competing as a substrate for activated TGs, Fn and other components of blood clots are crosslinked.

Inflammation and fibrosis of both the proximal vein and the vascular access site, as well as accelerated atherosclerosis, may be explained by the deposition of Fn in the vascular lumen. Fibronectin deposition on the vascular endothelia promotes platelet and white blood cell adherence, both of which may be aggravated in the absence of PLA₂ inhibition. Vascular deposits of Fn may also promote local deposits of fat, cholesterol and protein found in atherosclerotic plaque. Fibronection is known to be a major component of atherosclerotic plaque, as well as renal glomerular deposits associated with nephropathy and loss of primary and residual renal function. Therefore, UG administration may reduce or eliminate these problems by reducing inflammation and fibronectin deposition.

The preferred route of administration of UG would be intravenous infusion before, during or after dialysis.

Alternatively, the loss of endogenous UG may be prevented by addition of UG to the dialysis buffer or precoating the dialysis membrane with UG or both.

Organ Transplants

The term “organ” refers, for example, to solid organs, such as kidney, liver and heart, as well as bone marrow, cornea and skin.

There are two types of organ transplant rejection: acute and chronic. Acute rejection is an inflammatory process involving PLA₂ activity and infiltration by inflammatory cells that often destroys the graft.

Chronic rejection involves Fn-mediated fibrosis of the graft, including atherosclerosis confined to the graft. Thus, administration of UG may be used to treat or prevent both acute and chronic graft rejection.

The preferred route of administration is by injection.

Another aspect of organ transplantation is ischemia of the organ before removal from the donor, during transport and in the recipient, which contributes to acute rejection. Ischemia is known to result in elevated PLA₂ activity and tissue necrosis. Hence, UG could be used to prevent such ischemia. The preferred form of UG is as a perfusion liquid or storage buffer in which the ex vivo organ is preserved.

Prevention of Type I Diabetes

Type 1 diabetes arises from the destruction of pancreatic tissue by an autoimmune response. The pancreas normally secretes soluble PLA₂s and hUG into the circulation. Necrotic lesions have been reported in the pancreas of the uteroglobin knockout (KO) mouse of the present invention (herein referred to as the “UG KO mouse”).

In the absence of uteroglobin, the UG KO mouse exhibits similar pancreatic tissue destruction which could trigger an autoimmune response. Thus UG may be used to prevent or halt the slow progression of Type 1 diabetes. The preferred route of administration is by injection.

Prevention and Treatment of Nephropathy

Renal Fn deposits and fibrosis in the UG KO mouse are similar to Fn deposits and fibrosis in human nephropathies. Thus, UG administration may prevent or slow the progression of nephropathy in patients at risk, such as Type II diabetes.

Prevention and Treatment of Ocular Inflammation

Ocular inflammation, including uveitis, retinitis, and inflammation following surgery, is characterized by increased PLA₂ activity. Therefore, UG may be administered topically, intraocularly, or systemically to reduce ocular inflammation.

Arteriosclerosis

Arteriosclerosis is a fibrotic thickening of blood vessels throughout the body. It is initiated and/or mediated by Fn deposition on the walls of the vasculature. Atherosclerosis is a form of arteriosclerosis involving cholesterol deposition, in addition to Fn deposition. Therefore, UG may be administered to prevent or reduce arteriosclerosis.

Acute Renal Failure

Acute renal failure (ARF) is typically a consequence of remote organ inflammation, infection or direct trauma, which results in release and activation of soluble PLA₂ in the circulation. Damage to the kidneys during ARF can be quite severe, with acute tissue damage promoted by inflammation and may resolve into fibrosis of the kidney, leading to reduced kidney function in the long term. The anti-inflammatory and anti-fibrotic properties of UG are particularly relevant in the kidney as shown by the UG KO mouse.

The preferred route of administration is by injection or systemic administration.

Prevention and Treatment of Primary Tumor Growth

Tumorigenesis is a result of uncontrolled cell growth and invasion of surrounding tissues. The tumor suppressor activity of uteroglobin mediated by its cellular receptors is indicative of its potential as a prophylactic and/or therapeutic agent in the treatment of human cancer. Further, the development of tumors in aged uteroglobin deficient mice shows the physiological significance of long term depletion of uteroglobin in cancer.

The preferred route of administration is by injection or systemic administration.

Prevention and Treatment of Tumor Metastasis

The role of fibronectin deposition in tumor cell adhesion and tumor metastasis has been well characterized (Snyder, et al., “Fibronectin: Applications to Clinical Medicine” in CRC Critical Rev. Clin. Lab. Sci. 23(1): 15-34 (1985)). The ability of uteroglobin to prevent fibronectin aggregation and deposition in vivo shows the utility of uteroglobin in the prevention and treatment of human cancer metastasis.

The preferred route of administration is by systemic administration.

Prevention and Treatment of HIV Infection

Infection of human white blood cells by the human immunodeficiency virus (HIV) is mediated by at least two types of membrane bound HIV receptors. Uteroglobin could prevent infection of white blood cells by blocking one or more of the HIV receptors.

Therefore, exogenous human uteroglobin may be administered by injection or by systemic administration to patients with HIV or those exposed to HIV.

Stimulation of Hematopoiesis

Clinical conditions characterized by deficiencies of white and/or red blood cells may be treated with agents that stimulate hematopoiesis. The patient populations effected by such clinical conditions include those undergoing chemotherapy, dialysis, and patients with genetic anemias. Because human uteroglobin has been shown to be a growth factor for white blood cells (Aoki et al., 1996) and HAF is known to stimulate both red and white blood cell growth, human uteroglobin may be used to treat human anemias. All growth factors mediate their effects through membrane bound cellular receptors, and therefore, uteroglobin and its derivatives may be used to target the uteroglobin receptor(s) to stimulate hematopoiesis.

Preferred routes of administration include injection and systemic administration.

Overall, the following non-limiting list of conditions are those associated with inhibition of PLA₂ and/or fibronectin deposition and/or tumor suppression and/or UG receptor targeting, each of which are candidates for treatment or prevention by the method of present invention:

| | |
|---|---|
| Joint/Bone: | rheumatoid arthritis and sarcoma; |
| Autoimmune: | rheumatoid arthritis, multiple sclerosis, Type 1 diabetes, |
| | uveitis, psoriasis, systemic lupus erythematosus (SLE), and |
| | Crohn's disease; |
| Pancreas: | pancreatitis, sarcoma and carcinoma; |
| Peritoneum: | peritonitis, appendicitis, carcinoma and sarcoma; |
| Vascular/systemic: | septic shock; collagen vascular disease, |
| | arteriosclerosis, atherosclerosis, anaphylactic |
| | shock, schistosomiasis, trauma-induced shock, carcinoma, |
| | endothelioma and sarcoma; |
| Renal: | acute renal failure, bacterial infection of the kidneys, |
| | inflammation due to renal tumors, prevention of fibrosis |
| | resulting from chemotherapy or antibody therapy, |
| | prevention of diabetic nephropathy, prevention and/or |
| | treatment of idiopathic nephropathy, sarcoma and |
| | carcinoma; |
| Liver: | hepatitis, viral hepatitis, and cirrhosis, sarcoma, carcinoma; |
| Bladder: | cystitis, inflammation of the urethra, inflammation of the |
| | ureter, bladder inflammation such as interstitial cystitis, |
| | sarcoma and carcinoma; |
| Reproductive/female: | vaginitis, inflamed cervix, pelvic inflammatory disease, |
| | inflammation of the ovary (salpingitis), endometriosis, |
| | vaginal candidiasis, inflammation or fibrosis of the |
| | fallopian tubes, carcinoma and sarcoma; |
| Reproductive/male: | penile inflammation, prostate inflammation, inflammation |
| | of seminal tubules and vesicles, testicular inflammation, |
| | inflammation of vas deferens, epididymis, and prostate |
| | gland, carcinoma and sarcoma; |
| Ocular: | uveitis, retinitis, trauma, burn damage due to chemical or |
| | smoke, ocular inflammation due to CMV retinitis, |
| | conjunctivitis (bacterial infection), viral infection, ocular |
| | inflammation due to infectious agent, ocular inflammation |
| | following ocular surgery, including cataract removal, laser |
| | surgery, corneal transplant, tumor removal, ocular |
| | inflammation due to retinoblastoma (tumor), ocular |
| | inflammation due to radiation exposure, inflammation due |
| | to allergic response, sarcoma and carcinoma; |
| Heart: | Endocarditis, sarcoma and carcinoma |
| Lungs: | bronchial asthma, ARDS, pneumonia, idiopathic pulmonary |
| | fibrosis, pulmonary fibrosis resulting from chemotherapy |
| | (bleomycin, methotrexate), pulmonary fibrosis resulting |
| | from exposure to environmental chemicals (asbestos, |
| | cleaning fluids, pollutants, e.g. dioxin and PCB's in |
| | automobile exhaust), smoke inhalation, pulmonary |
| | inflammation during recovery from drowning, neonatal |
| | RDS, carcinoma and sarcoma; |
| Gut: | inflammatory bowel disease, colitis, Crohn's disease, |
| | direticulitis, neonatal necrotizing enterocolitis, |
| | inflammation due to an infectious agent, rotavirus, polio |
| | virus, HIV, stomach ulcers, gastro-esophageal reflux |
| | disease, tonsillitis, carcinoma and sarcoma; |
| Hemorrhoids; | |
| Transplants: | administration following transplant surgery for any |
| | organ or tissue to control inflammation or fibrosis |
| | and rejections; |
| Ears: | Otitis media, carcinoma and sarcoma; |
| Skin: | psoriasis, hives, allergic and dermatitis, scleroderma, |
| | contact dermatitis, chemical dermatitis (due to poison ivy, |
| | poison oak, and exposure to chemicals like PCB's, |
| | chlorine, ammonia, (cleaning agents, toxic agents)), |
| | carcinoma and sarcoma; |
| Spleen/Thymus: | Sarcoma and carcinoma; |
| Muscle: | Sarcoma and carcinoma; |
| Hemapoietic/Lymphatic: | Carcinoma and sarcoma; |
| Embryonic: | Carcinoma and sarcoma; and |
| Glandular | Carcinoma and sarcoma. |
| (endocrine glands): | |

Moreover, UG may be administered either alone or in combination with other active agents or compositions typically used in the treatment or prevention of the above-identified disease conditions. Such active agents or compositions include, but are not limited to steroids, non-steroidal anti-inflammatories (NSAIDs), chemotherapeutics, analgesics, immunotherapeutics, antiviral agents, antifungal agents, vaccines, immunosuppressants, hematopoietic growth factors, hormones, cytokines, antibodies, antithrombotics, cardiovascular drugs, or fertility drugs. Also included are oral tolerance drugs, vitamins and minerals.

The present invention relates to the use of UG in the prevention or treatment of PLA₂ and fibronectin associated conditions, and cancer and UG-receptor associated conditions. With regard to prevention of a disease condition, “prevention” refers to preventing the development of disease in a susceptible or potentially susceptible population, or limiting its severity or progression, whereas the term “treatment” refers to the amelioration of a disease or pathological condition.

UG may be administered to target a UG-receptor. Targeting of a UG receptor refers to inducing specific binding of a ligand to a receptor to mediate effects on cell growth.

UG may be administered intravenously or, in the case of treatment of neonatal RDS/BPD and adult RDS, in the form of a liquid or semi-aerosol via the intratracheal tube. Other viable routes of administration include topical, ocular, dermal, transdermal, anal, systemic, intramuscular, slow release, oral, vaginal, intraduodenal, intraperitoneal, and intracolonic. Such compositions can be administered to a subject or patient in need of such administration in dosages and by techniques well known to those skilled in the medical, nutritional or veterinary arts taking into consideration such factors as the age, sex, weight, and condition of the particular subject or patient, and the route of administration. The compositions of the present invention may also be administered in a controlled-release formulation. The compositions can be co-administered or sequentially administered with other active agents, again, taking into consideration such factors as the age, sex, weight, and condition of the particular subject or patient, and, the route of administration.

Examples of compositions of the invention include edible compositions for oral administration such as solid or liquid formulations, for instance, capsules, tablets, pills, and the like liquid preparations for orifice, e.g., oral, nasal, anal, vaginal etc., formulation such as suspensions, syrups or elixirs; and, preparations for parenteral, subcutaneous, intradermal, intramuscular or intravenous administration (e.g., injectable administration), such as sterile suspensions or emulsions. However, the active ingredient in the compositions may complex with proteins such that when administered into the bloodstream, clotting may occur due to precipitation of blood proteins; and, the skilled artisan should take this into account.

In such compositions UG may be in admixture with a suitable carrier, diluent, or excipient such as sterile water, physiological saline, glucose, DMSO, ethanol, or the like. UG can be provided in lyophilized form for reconstituting, for instance, in isotonic aqueous, saline, glucose, or DMSO buffer. In certain saline solutions, some precipitation of rhUG has been observed; and this observation may be employed as a means to isolate inventive compounds, e.g., by a “salting out” procedure.

Further, the invention also comprehends a kit wherein UG is provided. The kit can include a separate container containing a suitable carrier, diluent or excipient. The kit can include an additional agent which reduces or alleviates the ill effects of the above-identified conditions for co- or sequential-administration. The additional agent(s) can be provided in separate container(s) or in admixture with UG. Additionally, the kit can include instructions for mixing or combining ingredients and/or administration.

The invention also contemplates a method for treating or preventing cancer characterized by a deficiency of endogenous functional UG, which comprises administering to a patient in need of such treatment a compensating amount of UG. The term “compensating amount” means an amount of UG required to bring the local pulmonary or systemic concentration of total UG (endogenous functional UG and exogenous UG) to within its normal range. More specifically, the normal range for local pulmonary concentration of endogenous UG is about >50 micrograms UG/milligram albumin or >50 micrograms/liter. The normal range for serum UG concentration is >15 micrograms/liter. Moreover, excess uteroglobin may be administered in an amount sufficient to saturate both soluble and insoluble (membrane bound) uteroglobin binding moieties in the body, which amount may exceed a compensating amount of uteroglobin as defined above, such that the circulating level of uteroglobin is approximately 2-200 times above normal.

The compositions of the invention comprise native and/or recombinant hUG in an amount effective to achieve the intended purpose, namely increased plasma or tissue levels of UG to produce the desired effect of tumor suppression and/or binding of fibronectin to mitigate its role in metastasis. The compositions comprise an effective amount of substantially pure native and/or recombinant human UG, in association with a pharmaceutically acceptable carrier or diluent. Uteroglobin may exist in either the reduced or monomeric form, or both.

Uteroglobin may be administered in an amount of a single bolus of 20 ng/kg to 500 mg/kg, in single or multiple doses, or as a continuous infusion of up to 10 grams.

The term “tumor suppressing effective amount” as used herein means the amount of UG which suppresses tumors and which prevents or reduces tumor metastasis in the tissue or body of the patient. The term “fibronectin binding effective amount” means that amount of UG which binds fibronectin to reduce aggregation and/or deposition thereof, and prevent or reduce tumor metastasis. Similarly, the term “hematopoiesis-stimulating effective amount” means that amount of uteroglobin which can be administered to stimulate red and white blood cell growth. Moreover, the term “anti-HIV effective amount” is that amount of uteroglobin sufficient to block one or more HIV receptors. Typically, the amount of UG administered to adults for the treatment of cancer will be single boluses of 0.2 μg/kg to 500 mg/kg or up to several grams administered over an extended period of time. For neonates, in the treatment of neonatal RDS, the range will typically be 50 nanograms/kg to 100 mg/kg in single boluses or up to 10 grams administered continuously over an extended period of time. Effective and safe rates of continuous infusion are between 50 ng/kg/hour to 500 mg/kg/hour.

Moreover, the present invention provides a method of purifying a uteroglobin receptor(s) by affinity chromatography using rhUG bound to a solid support. The method comprises contacting a sample, e.g. bovine heart, spleen, trachea, lung, liver and aorta, which may be solubilized or partially purified prior to affinity chromatography, with a solid support having uteroglobin (or a fragment or derivative thereof, or a UG-like protein or UG-receptor ligand) coupled thereto. UG may be bound covalently to the solid support, i.e. CNBr-activated Sepharose 4B, or by any method or to any solid support known to those in the art. The UG-receptor protein is then eluted from the solid support using a suitable buffer.

Further, the present invention provides a method of preparing reduced rhUG. The method of the present invention consists of contacting oxidized or partially oxidized rhUG with a reducing agent, e.g., dithiothreitol or B-mercaptoethanol, for a time and temperature sufficient to reduce rhUG, e.g. at 37° C. for 15 minutes. In a preferred embodiment, the method of the present invention yields reduced, monomeric rhUG. One of ordinary skill in the art will appreciate that any suitable reducing agent or combination of reducing agents may be used for an appropriate time and at a suitable temperature sufficient to reduce rhUG, as evidenced by HPLC, SDS-Page, or other suitable detection methods.

Additionally, the uteroglobin receptor may be purified by standard techniques known to those skilled in the art and used to screen compounds, peptides or proteins which are uteroglobin structural analogs and/or UG-receptor ligands. In this regard, the purified uteroglobin receptor may also be used in a kit for screening for uteroglobin structural analogs and/or UG-receptor ligands. Such a screening method comprising contacting a sample comprising one or more compounds, peptides and/or proteins with a purified uteroglobin receptor and detecting a binding interaction between one or more of the components in the sample and the uteroglobin receptor. Such binding interactions, e.g. ligand-receptor interactions, may be detected by, for example, changes in the UV spectra for the receptor, or by any other method known to those skilled in the art, and are indicative of the presence of a uteroglobin structural analog and/or a UG-receptor ligand in the sample.

Finally, the purified uteroglobin receptor(s) may be used to generate antibodies against the receptor. Such antibodies may be used to stimulate and activate uteroglobin receptors and may be generated used standard techniques known to those skilled in the art, for example, immunizing mice with purified uteroglobin receptor, preparing hydridomas, and screening for antibodies to uteroglobin receptor(s). See, for example, Sambrook et al., “Molecular Cloning: A Laboratory Manual, 2d Ed.” Cold Spring Harbor Laboratory Press, NY, 1989.

### EXAMPLES

The invention will now be further described with reference to the following non-limiting examples. Parts and percentages are by weight unless otherwise stated.

### Example 1

### In Vivo Experiments

Recombinant human UG was obtained by the method of Mantile et al. (1993).

One male and one female of the species P. cynocephalus, weighing approximately 400 grams each were delivered by C-section at 142 days of gestation. This is an established model of RDS (Coalson, J. J., et al. Baboon Model of BPD. II: Pathologic features. Exp. Mol. Pathol. 37: 355-350 (1982)).

After delivery, the infants were anesthetized with ketamine (10 mg/kg) and intubated with a 2.5 mm diameter endotracheal tube. Blood gases and pressure were monitored via an arterial line placed by percutaneous injection into the radial artery. A deep venous line was placed percutaneously into the saphenous vein through which fluids, antibiotics, and drugs were administered. Animals were maintained on servo-controlled infrared warmers and ventilated with a standard time-cycled, pressure-regulated ventilator with humidifiers maintained at 36-37° C. Initial setting were FiO₂1.0, rate 40/min., I/E ratio 1:1.5, positive end expiratory pressure (PHEP) at 4 cm H₂O, and peak inspiratory pressure (PIP) as required for adequate chest excursion. FiO₂ was kept at 1.0 and PIP was regulated to maintain PaCO₂ at 40±10 torr. Blood gases, hematocrit, electrolytes, prothrombin time, partial thromboplastin time and dextrostix were monitored hourly. Blood drawn for studies was replaced volumetrically with heparinized adult baboon blood. Intravenous fluids were administered with electrolytes at 10 cc/kg/hr and were increased as needed when heart rate exceeded 180 beats/min. Sodium bicarbonate (2 meq/kg) was administered when the base deficit exceeded -10. Ampicillin (50 mg/kg/day in two divided doses) and Gentamicin (5 mg/kg/day in two divided doses) was given continuously for the duration of the experiment.

One animal received surfactant plus PBS (treatment no. 1), and the second animal (treatment no. 2) received surfactant plus two doses of 1 mg/kg of rhUG. Both surfactant and rhUG were administered directly to the lungs through the endotracheal tube. The surfactant used was Survanta (Ross Labs), a surfactant preparation derived from bovine lung tissue, containing surfactant apoproteins B and C in addition to phospholipids. The first dose of rhUG was given with the surfactant and the second administered four hours after the first. The animals were monitored for arterial blood gases, electrolytes and EKG. They were sacrificed 50 hours after the initiation of surfactant therapy. The lungs were lavaged at 24 and 48 hours with PBS containing protease inhibitors (PMSF, 10 μg/ml leupeptin, 10 μg/ml of pepstatin and bacitracin). They were frozen at −80° C. until assayed for PLA₂ activity. Total proteins were determined by Bradford method (BioRad). The PLA₂ activity in the lung lavages were measured according to Levin et al. (1986; supra) and are presented in the following Table.

TABLE 3
Results of In Vivo Testing of UG

| | | Lung lavage PLA₂ activity |
|---|---|---|
| Treatment # | Time | (ccpm/10 μg protein |
| 1 | 24 hr | 3030 |
| | 48 hr | 2607 |
| 2 | 24 hr | 1739 |
| | 48 hr | 996 |

The data given above are the mean of two determinations. The results show that endotracheal administration of rhUG inhibits PLA₂ in vivo. The animals which received surfactant and rhUG had an appreciably lower PLA₂ activity in their lung lavage fluid compared with the animals that received surfactant without rhUG. The data confirm that administration of rhUG in conjunction with surfactant is beneficial in protecting surfactant phospholipids.

### Example 2

### Inhibition of Hydrolysis of Artificial Surfactant by Soluble PLA, In Vitro

RhUG inhibits hydrolysis of artificial surfactant by soluble PLA₂s in vitro. Survanta is an artificial surfactant derived from bovine lung and is used to treat pre-term neonates with RDS and adults with RDS (ARDS). Hydrolysis of Survanta by a Group I soluble PLA₂, i.e. porcine, pancreatic PLA₂ (Boehringer Mannheim) is characterized by its ability to compete as a substrate with a fluorescent phosphatidylcholine substrate (Cayman Chemicals), generating arachidonic acid as a product.

Survanta is a substrate for in vitro degradation by Group I soluble PLA₂s. Survanta is rapidly degraded in vitro by PLA₂s found in the extracellular fluids of a human lung. RhUG inhibits degradation of Survanta in vitro.

### Example 3

### Construction of UG Knockout Mouse

A transgenic UG KO mouse was created for the purpose of determining the role of uteroglobin in mammalian physiology, as well as to generate a model for UG as a therapeutic in several inflammatory clinical conditions. The first step was to construct an appropriate DNA vector with which to target and interrupt the endogenous murine uteroglobin gene. The 3.2 kb BamHI-EcoRI DNA fragment containing exon 3 and flanking sequences of the uteroglobin gene from the 129/SVJ mouse strain (Ray, 1993) were subcloned into the corresponding sites of the pPNW vector as described in Lei et al (1996). A 0.9 kb fragment containing part of exon 2 and its upstream sequence was amplified by PCR (with primers Primer-L (from Intron 1): 5′-TTC CAA GGC AGA ACA TIT GAG AC-3′; Primer-R (from Exon 2): 5′-TCT GAG CCA GGG TTG AAA GG C-3′) with NotI and XhoI restriction sites engineered into the termini for directional subcloning into the gene targeting vector. In this construct, 79 bp of Exon 2 encoding 27 amino acids were deleted. The PCR fragment was placed upstream of the gene encoding neomycin resistance in pPNW, generating the gene targeting vector, pPNWUG. The vector is shown in FIG. 2A, in which the PGK-neo cassette interrupts the uteroglobin gene, disrupting the protein coding sequence.

The pPNWUG gene targeting vector was linearized with NotI and electroporated into ES R₁ cells according to Nagy, A., et al. PNAS 90:8424 (1993). Gancyclovir and G-418 selection of the electroporated cells yielded 156 clones. Southern (DNA) blot analysis identified a 5.1 kb HindIII fragment of the wild-type uteroglobin allele and an additional 8.2 kb HindIII fragment resulting from homologous recombination in three out of the 156 clones, shown in FIG. 2B. These ES R1 clones were injected into C57BL/6 blastocysts according to Capecchi, Science 244: 1288 (1989). Two different lines of mice, descended from different chimeric founders, were generated. Heterozygous offspring (UG^{+/-}) carrying the targeted uteroglobin gene locus were mated and the genotypes of the progeny were analyzed by PCR shown in FIG. 2C, as well as Southern blot, shown in FIG. 2D.

### Example 4

### Verification of UG Gene Knockout and Absence of Murine UG (mUG) Protein

In order to verify that the homozygous knockout mice (UG^{-/-}) did not possess any detectable mUG, the uteroglobin gene-targeted mice were tested for expression of UG-mRNA and mUG protein in several organs including the lungs. An experimental protocol was approved by the institutional animal care and use committee. Total RNAs were isolated from different organs of UG^{+/+}, UG^{+/-}, and UG^{-/-} mice. The reverse transcribed-polymerase chain reaction (RT-PCR) was used to detect mUG-mRNA. Target molecules were reverse transcribed using a mUG-specific primer, mPr (5′-ATC TrG CTT ACA CAG AGG ACT TG-3′), and the cDNA generated was amplified using PCR primers mPr and mPl (5′-ATC GCC ATC ACA ATC ACT GT-3′). The PCR product was hybridized with an oligonucleotide probe, mPp (5′-ATC AGA GTC TGG TTA TGT GGC ATC C-3′) derived from exon-2 of the UG gene sequence. The primers and the probe used in mouse GAPDH RT-PCR are as follows: mGAPDH-r (5′-GGC ATC GAA GGT GGA AGA GT-3′); mGAPDH-1 (5′-ATG GCC TTC CGT GTT CCT AC-3′); mGAPDH-p (5′-GAA GGT GGT GAA GCA GGC ATC TGA GG-3′). FIG. 2E shows that mUG-mRNA was detected in the lungs of UG^{+/+}, and UG^{+/-}, but not UG^{-/-} mice. Similar data (not shown) show that mUG-mRNA is not present in either the prostate or uteri of UG^{-/-} mice, but is present in the mice with an intact uteroglobin gene.

Immunoprecipitation and Western blot analyses of mUG protein in the lungs yielded similar corroborative results, shown in FIG. 2F. Tissue lysates from the kidneys, liver, and the lungs of the UG^{+/+} and UG^{-/-} mice were prepared by homogenizing in a buffer (10 mM Tris-HCl, pH 7.5, 1% Triton X-100, 0.2% deoxycholate, 150 mM NaCl, 5 mM EDTA) containing 2 mM phenylmethylsulfonyl fluoride and 20 μg/mL each of aprotinin, leupeptin, and pepstatin A. The homogenates were centrifuged at 17,500× g for 30 min at 4° C. and immunoprecipitated as described (E. Harlow and D. Lane, Antibodies; a laboratory manual, 1st Ed. Cold Spring Harbor Laboratory Press, Cold Spring Harbor, N.Y., 1988) by incubating tissue lysates or plasma proteins (1 mg/mL) with rabbit antibody against murine Fn (1:100 dilution). Co-immumoprecipitation of purified murine Fn (mFn) and rhUG (Mantile, G, et al., J. Biol. Chem. 267: 20343 (1993)) was performed by incubating equimolar concentrations of mFn with rhUG in the presence of 10% glycerol, 50 mM Tris-HCl, pH 7.5, 250 mM NaCl, 4.3 mM sodium phosphate at 4° C. for 1 hr., followed by adding anti-mFn antibody (1: 100 dilution). Equal amounts of extracted tissue proteins (30 μg) or immunoprecipitates were resolved either on 4-20% or 6% SDS-polyacrylamide gels under reducing conditions, followed by Western blotting with rabbit antibodies against either murine Fn (1:2000 dilution) or UG (1:2000 dilution). No mUG was detected in tissues or fluids from the UG^{-/-} mice, while tissues from UG^{+/+} and UG^{+/-}0 mice did contain the mUG protein.

Finally, histopathological analyses of the lungs of UG^{-/-}, only, lacked mUG-specific immunostaining in bronchiolar epithelial cells. Lung tissues from UG^{-/-}, UG^{+/-} and UG^{+/+} mice were fixed in Bouin's fluid or in 10% neutral buffered formalin fixatives, embedded in paraffin and sectioned at 4-6 microns. They were stained with hematoxylin and eosin (H & E). Selected tissues were stained by Masson's trichrome method for collagen detection, PTAH for fibrin, or Congo Red for amyloid protein. For immunohistochemical detection of mUG and mFn, the Vectastain rabbit Elite ABC kit (Vector Laboratories) was used. The rabbit antibody (CytImmune) to mUG was raised by using a synthetic peptide (Peptide Technologies, Inc.) corresponding to mUG amino acid sequence (Lys28 to Thr49, specifically KPFNPGSDLQNAGTQLKRLVDT). The rabbit antibody to mFn (GIBCO BRL) was used at a dilution of 1:1000, and the antibody to mUG was used at 1:500.

These three sets of results confirm that the homozygous uteroglobin knockout mouse, UG^{-/-}, lacks mUG protein, or any detectable piece of the protein.

### Example 5

### Phenotype of Uteroglobin Knockout Mouse

Of the 179 mice born to crosses of UG^{+/-} mice, 46 (26%) were of the +/+, 90 (50%) of the +/− and 43 (24%) of the UG^{-/-} genotype, indicating that the disrupted mUG locus is inherited in a Mendelian fashion and that UG^{+/+}, UG^{+/-}, and UG^{-/-} mice were equally viable at birth. However, UG^{-/-} mice exhibited a novel phenotype in which they developed a progressive illness characterized by cachexia, heavy proteinuria, and hypocalcemia associated with profound weight loss. Proteinuria is a condition in which abnormally high levels of albumin and other serum proteins are excreted in the urine. It is indicative of glomerular dysfunction and renal failure. Histopathological examination of the kidneys of affected animals (as described above for lungs) revealed the fulminant renal glomerular disease shown in FIG. 3. Compared with the glomeruli of the UG^{+/+} mice, those of UG^{-/-} mice were hypocellular and had massive eosinophilic proteinaceous deposits. The time course of the fatal renal disease in UG^{-/-} mice was either early onset (4-5 week period) or late onset (10 month period). Those UG^{-/-} mice that initially appeared healthy at 4 weeks of age had focal glomerular deposits at two months of age. At about 10 months, these mice had extreme cachexia similar to that of the mice dying of early onset disease. Heterozygotes had a milder form of the renal disease observed in UG^{-/-} mice. Histopathology of the kidneys of mice with late onset disease showed not only severe glomerularopathy as in the early onset disease, but also had marked fibrosis of the renal parenchyma and tubular hyperplasia (see FIG. 3). Although the predominant pathology in the UG^{-/-} mice was found in the kidneys, histopathological studies also uncovered occasional focal areas of necrosis in the pancreas which appeared to be vascular oriented. Moreover, focal areas in the thymus and in the spleen structures suggestive of apoptotic bodies were also found. Interestingly, the pancreas expresses the mUG gene, and this organ is also a rich source of group-I extracellular PLA₂; since this is primarily a digestive enzyme, its activation may cause tissue injury.

Because uteroglobin proteins have has been reported to have immunomodulatory and anti-inflammatory properties and because reactive amyloidosis is known to occur in response to inflammation, it was likely that the glomerular deposits in the mUG-null mice were amyloid proteins. Reactive amyloidosis is characterized by the deposition of amyloid protein and immune complexes. The identity of the renal deposits in the UG^{-/-}0 mice was established by immunohistochemistry of kidney sections. Kidney sections from UG^{-/-} and UG^{+/+} mice were stained with Congo red and examined under the polarized light. Amyloid proteins yield a positive birefringence in this test; however, the glomeruli of UG^{-/-} mice were clearly negative. Immunofluorescence studies for the presence of IgA, IgG or IgM-immunocomplexes in the glomeruli of UG^{-/-}0 mice and immunohistochemical analyses for the presence of major amyloid proteins were also negative. Thus, the glomerular deposits of UG^{-/-} mice contained neither amyloid proteins nor immunocomplexes, and therefore, do not appear to be the result of an inflammatory response.

### Example 6

### Detection of Fn and Collagen in UG^{-/-} kidneys

The kidney deposits of UG^{-/-} mice were examined by transmission electron microscopy to elucidate their structure and morphology. A kidney from a UG^{-/-} mouse, with glomerular lesion, was fixed in formalin and embedded in epoxy resin. Thin sections were stained with uranyl acetate and lead citrate for examination under the electron microscope. Photomicrographs were taken either at 6000× or at 60,000×. The deposits contained primarily two types of fibrillar structures: one type of long and striated fibrils which are relatively infrequent, the other short and diffuse which are more abundant (FIGS. 3E and 3F). Because ECM proteins, such as collagen and fibronectin, produce similar fibrillar structures, the glomerular deposits in UG^{-/-} mice may contain these proteins.

The glomerular deposits were next analyzed by immunofluorescence using anti-mFn antibody. Formalin-fixed tissue sections were used for immunofluorescence using a rabbit anti-mFn and FITC-conjugated goat anti-rabbit IgG. Similarly, immunofluorescence studies using antibodies specific for mFn, collagen I and III, vitronectin, laminin and osteopontin were also done. Epifluorescence was photographed using a Zeiss Axiophot microscope. Fn-specific immunofluorescence in the renal glomeruli of wild-type mice was virtually undetectable (FIG. 3G), that in the glomeruli of UG^{-/-} littermates was intense (FIG. 3H). When Masson's trichrome staining was used, the glomeruli of UG^{+/+} mice were negative (FIG. 31) and those of UG^{-/-} (FIG. 3J) mice were positive, suggesting the presence of collagen in the glomerular deposits. Immunofluorescence, using collagen I and collagen III-specific antibodies confirmed these results. Because Fn is known to interact with other extracellular matrix (ECM) proteins, we also tested for the presence of laminin, vitronectin and osteopontin in the glomeruli of UG^{+/+} and UG^{-/-} mice by immunohistochemistry, the results of which were negative.

### Example 7

### Kidneys of UG^{-/-}0 Mice Do Not Overproduce Fn

In order to determine whether excessive production of Fn may account for its deposition in the renal glomeruli, we assessed the relative amount of Fn-mRNA in the kidneys, lungs, and the liver of UG^{-/-} and UG^{+/+} mice by RT-PCR and densitometry. The results indicate that relative amounts of Fn-mRNA were essentially identical in both UG^{+/+} and UG^{-/-} animals. Thus, over-production of Fn-mRNA was not a likely cause of Fn-deposition in the glomeruli of UG^{-/-} mice. We then compared the Fn-protein in the plasma, kidneys, and the liver of UG^{-/-} and UG^{+/+} mice by SDS-PAGE under reducing conditions, and Western blotting. In the plasma, kidneys and the liver of wild-type mice only 220-kD Fn species could be detected; however, whereas the plasma and the liver lysate of UG^{-/-} mice had the 220-kD Fn band, the kidney lysates contained another distinct, covalently linked, multimeric Fn-band (FIG. 4A).

### Example 8

### Elevated Serum PLA, Activity in UG^{-/-} Mice

Based upon current concepts, critical initial steps in Fn matrix-assembly and fibrilogenesis, at least on the cell surface, are thought to involve integrin activation and Fn self-aggregation. Because UG is a potent inhibitor of soluble phospholipase A₂ (sPLA₂), a key enzyme in the inflammatory pathway, the lack of mUG in UG^{-/-} mice may contribute to the development of glomerulonephritis, an inflammatory renal disease. Thus, we measured PLA₂ activity in the serum of age, sex and weight-matched UG^{+/+} (n=3) and UG^{-/-} mice (n=3). The animals were sacrificed and serum PLA₂ activities of each sample were measured in triplicate using a PLA₂-assay kit (Caymen Chemical) according to the instructions of the manufacturer. Protein concentrations in the sera were determined by Bradford assay (Bio Rad) and specific activities of PLA₂ were calculated. The specific activities (μmol/min/mg protein) of serum PLA₂ of UG^{-/-} mice [36+3.3 (SEM)] were significantly higher (p<0.05) than those of UG^{+/+} mice [18+2.8 (SEM)]. These results raised the possibility that higher PLA₂ activity may lead to increased lysophosphatidic acid (LPA) production and consequently promote integrin activation and Fn-self aggregation in UG^{-/-} mice.

### Example 9

### Interaction of Uteroglobin and Fibronectin In Vitro

To further understand how uteroglobin may prevent Fn self assembly, the ability of rhUG to disrupt mFn-Fn interaction in vitro was determined. Equimolar concentrations of rhUG and mFn were incubated to allow any protein binding or other interactions, then immunoprecipitated with anti-Fn-antibody, and the immunoprecipitates were resolved by SDS-PAGE under reducing conditions. Western blotting, as previously described, with either mFn or mUG antibody detected each protein, respectively. The results show that fibronectin co-immunoprecipitated with rhUG (FIG. 4B). To confirm these results, the ¹²⁵I-rhUG was incubated with mFn and the complexes resolved by electrophoresis, using a 6% polyacrylamide gel under non-denaturing and non-reducing conditions (FIG. 4C). Detection of a Fn-UG heteromer in the autoradiogram (lane 2) showed that soluble Fn interacts with UG in vitro. To ascertain whether Fn-UG heteromerization takes place in vivo, plasma of UG^{+/+} and UG^{-/-} mice was immunoprecipitated with an anti-mFn antibody that does not crossreact with rhUG (FIG. 4D). Anti-mFn antibody co-precipitated both mFn and rhUG from the plasma of UG^{+/+}, but not from UG^{-/-} mice, suggesting that Fn-UG heteromers are present in the plasma of UG^{+/+} mice. Therefore, the Fn-UG complex is not simply an artifact formed in vitro but occurs naturally in the serum.

To determine the specificity and affinity of UG binding to Fn, we incubated ¹²⁵I-Fn with unlabeled Fn in the presence and absence of UG. Any complexes were affinity-crosslinked with disuccinimidyl suberate (DSS). Using 24-well plates coated with human Fn (hFn) (Collaborative Biomedical Products), 3 μl of ¹²⁵I-Fn (Sp. Act. 6 mCi/mg: ICN Biomedicals) was incubated in the absence and presence of either UG or Fn (10⁻¹²-10⁻⁶ M) in 500 μl HBSS at room temperature for 2 hr. SDS-PAGE and Western blotting of all Fn with UG antibody failed to detect any UG contamination. The radiolabeled complex was washed twice with PBS, solubilized in 1 N NaOH, neutralized with 1 N HC1, and radioactivity was measured by a gamma counter. In a separate experiment 125I-hFn (3 μl) was incubated with 20 μl (1 mg/ml) of mouse Fn in 40 μl of HBSS, pH 7.6 in the absence or presence of increasing concentrations of reduced rhUG (5-500 μg) at room temperature for 2 hours. The samples were crosslinked with 0.20 mM DSS at room temperature for 20 min., boiled in SDS-sample buffer for 5 min., electrophoresed on 4-20% SDS-polyacrylamide gel and autoradiographed. In the absence of UG, ¹²⁵I-Fn formed a high molecular weight, radioactive complex with unlabeled Fn, but in the presence of UG the formation of Fn-Fn aggregates was inhibited in a manner dependent upon the UG concentration (FIG. 4E).

To determine whether there is any difference between the binding affinities of Fn for UG and that of Fn for itself binding experiments were performed in which ¹²⁵I-Fn was incubated with unlabeled Fn and immobilized on multiwell plates together with varying concentrations of UG. In separate experiments, binding studies of ¹²⁵I-Fn with unlabeled, immobilized Fn using various concentrations of unlabeled soluble Fn, were also done. The Scatchard analyses of the data from both types of binding experiments yielded straight lines with dissociation constants (kds) of 13 nM for UG binding to Fn and 176 nM for Fn binding to itself. These results suggest that, due to a relatively higher binding-affinity of UG for Fn, UG effectively counteracts Fn self-aggregation. Affinity-crosslinking experiments in which radio-iodinated (¹²⁵)-collagen I was incubated with unlabeled Fn in the absence or presence of UG, were also done as described above for Fn. Fifteen μl of either denatured or non-denatured ¹²⁵I-collagen I (Sp. Act. 65.4 mCi/mg) were incubated with Fn in presence or absence of reduced UG (250 μg), affinity crosslinked, electrophoresed and autoradiographed. The results indicate that UG counteracts the formation of high molecular weight ¹²⁵I-collagen-Fn aggregates (FIG. 4F).

### Example 10

### In Vivo Inhibition of Glomerular hFn Deposition by rhUG

To test whether rhUG protects the renal glomeruli from Fn accumulation, soluble human Fn (hFn) alone, or hFn mixed with equimolar concentrations of rhUG, was administered intravenously to UG^{+/+} and to apparently healthy UG^{-/-} littermates.

Human Fn (500 μg/150 μl PBS) was administered in the tail vein of two-month old, approximately 22 g, UG^{+/+} and apparently healthy, UG^{-/-} mice. Similarly, the control mice were injected with a mixture of 500 μg of hFn either with equimolar concentrations of rhUG or albumin in 150 ul PBS. Twenty-four hours after the last injection, the mice were sacrificed and various organs were fixed in buffered formalin. The histological sections of the kidneys and other organs were examined by immunofluorescence with a monospecific anti-hFn antibody (GIBCO BRL; clone 1) and FITC conjugated rabbit anti-mouse IgG (Cappel). In a separate experiment, UG^{+/+} mice were injected with 1 mg of hFn alone in 150 μl PBS daily for 3 consecutive days.

The rationale for injecting human Fn was to be able to discriminate between endogenous murine Fn and the administered hFn. The method of intravenous administration and immunohistochemical detection of hFn in various tissues have been described.

Human Fn immunofluorescence in the glomeruli of wild-type UG^{+/+} mice injected with either a mixture of hFn and rhUG (1:1 molar ratio) or with hFn alone was similar (FIGS. 5A and 5B). However, the UG^{-/-} mice injected with a mixture of hFn and UG showed little hFn-specific immunofluorescence in the glomeruli (FIG. 5C), while those receiving Fn alone exhibited higher intensity immunofluorescence (FIG. 5D). Administration of a mixture of hFn and BSA, as a control, yielded no protective effect.

To determine whether this UG protective effect could be overcome by injecting larger quantities of Fn in UG^{+/+} mice, we injected 1 mg of hFn per animal daily for three consecutive days. Although intravenous administration of hFn to UG^{+/+} mice at lower doses (500 μg/animal) was not effective in causing any appreciable glomerular deposition (FIG. 5A), the administration of higher doses (3 mg/animal) led to a significant accumulation. Thus, UG prevents glomerular Fn-deposition, and UG^{+/+} as opposed to UG^{-/-} mice have a higher threshold for the accumulation of soluble Fn, due to the presence of endogenous UG.

### Example 11

### Inhibition of Fibrilogenesis and Fn Matrix Assembly by rhUG in Tissue Culture Cells

To determine whether UG prevents Fn-fibrilogenesis and matrix assembly in a typical in vitro tissue culture assay, mouse embryonic fibroblasts were cultured in medium containing either soluble hFn alone or a mixture of equimolar concentrations of hFn and rhUG. Fn matrix assembly and fibrilogenesis in cultured cells (CRL6336, ATCC) were determined as described. The level of fibrilogenesis seen in the cells of cultures treated with hFn alone was much higher (FIG. 5E) compared to those which received a mixture of hFn and rhUG (FIG. 5F).

### Example 12

### Detection of UG-Fn Complexes in Clinical Samples

Detection of UG-Fn complexes in clinical samples of bodily fluids such as serum, BAL fluids, and sputum is important in determining the role of this complex in human disease. A solution phase diagnostic assay for the detection of UG-Fn complexes is developed and the assay format is shown in FIG. 6. The capture antibody, covalently linked to a solid support, is a monospecific rabbit polyclonal raised against the human protein. The solid support may be a bead, such as a magnetic bead, a tube, or an ELISA plate. The solid support affords the flexibility of performing wash steps after each binding reaction in order to obtain more consistent results with a variety of sample types. The detection antibody is specific for Fn, and available from a number of commercial sources. An anti-IgG antibody, conjugated to an enzyme such as horse radish peroxidase (HRP), is then used to detect the anti-Fn IgG at the end of the molecular chain in a standard enzymatic reaction in which the enzyme substrate is converted to a chromogenic or fluorogenic compound that is quantitated with a spectrophotometer or fluorimeter (Amersham). The detection limit for this assay is 500 μg of UG-Fn complex per ml of sample fluid.

### Example 13

### Uteroglobin Deficiencies

A transient but acute deficiency of hUG is created by the blood-cleansing technique known as clinical dialysis, including hemodialysis, peritoneal dialysis and continuous dialysis (CRRT). All forms of clinical dialysis involve the use of a semi-permeable membrane to filter toxic bodily waste products, including chemical metabolites such as urea, and small proteins such as beta2-microglobulin, out of the blood.

UG is an extremely compact protein, known for its anomalous migration in SDS-PAGE, corresponding to a molecular weight of approximately 10-13 kDa, despite its true molecular weight of 15.7 kDa. Therefore, the UG dimer was expected to behave as a 10-13 kDa protein in dialysis experiments. Surprisingly, it was found that the dimer is so compact that it passed through an 8.0 kDa MWCO dialysis membrane. UG also passed through a 14.0 kDa MWCO dialysis membrane.

The composition of the dialysis membranes used in these examples are similar, if not identical, to the composition of the majority of membranes manufactured and used for clinical dialysis. They consist of regenerated cellulose or cellulose acetate.

For this experiment, 1.0 ml aliquots of two partially purified (one >90% pure and one approximately 70% pure) rhUG cell lysates, with no buffer additives, were dialyzed against 1000 mls of unbuffered 50 mM ammonium acetate, using three sizes of dialysis tubing: 3.5 kDa, 8.0 kDa, and 14.0 kDa (Spectra/Por; Thomas Scientific). There were four changes of buffer for each sample over a 48 hour time period, all done at room temperature (about 25-27° C.). The appearance of each dialysis sample changed from a clear yellow to a clear, colorless liquid. Dialysis tubing was checked for leaks at the beginning and end of the process by brief application of pressure directly to the tubing (squeezing) and observation of any leaks, of which there were none. Tubing was double clamped at either end to further insure against leaks.

FIG. 7 shows the SDS-PAGE analysis of these results. The 90% pure pre-dialysis sample is shown in lane 7 and 8 next to the three post-dialysis samples in lanes 1, 2, and 3. The UG dimer is no longer present in the lanes representing the samples dialysed with 8.0 kDa MWCO membranes. These results were later confirmed with different batches of partially purified UG preparations.

### Example 14

### Affinity Crosslinking of hUG-binding Proteins to Tumor Cells

Previous work has shown that homodimeric, fully oxidized rhUG binds to a 190 kDa binding protein found in some types of tumor cells (Leyton et al, 1994; Kundu et al, 1996). The current example shows that reduced rhUG (later shown to be monomeric) binds to the 190 kDa UG-binding protein, as well as to a 49 kDa form and a ˜32 kDa form. It also shows that the presence of these UG-binding proteins correlates with the ability of exogenous rhUG to mediate a non-invasive phenotype in these tumor cells (NIH 3T3, mouse mastocytoma, sarcoma, and lymphoma). The absence of these UG-binding proteins correlates with persistence of the invasive phenotype in the presence of exogenous rhUG (fibrosarcoma). The following table gives new data demonstrating this phenotype in an ECM-invasion assay previously described (Kundu et al, 1996). An irrelevant protein control, myoglobin, was used to show that the effect is specific to rhUG.

| | | Invasion |
|---|---|---|
| Cell Type | Treatment* | (% Control)** |
| NIH 3T3 | None | 100 |
| | rhUG | 18 |
| | Myoglobin | 97 |
| Mastocytoma | None | 100 |
| | rhUG | 23 |
| Sarcoma | None | 100 |
| | rhUG | 21 |
| Lymphoma | None | 100 |
| | rhUG | 25 |
| Fibrosarcoma | None | 100 |
| | rhUG | 97 |

Briefly, confluent cells (NIH 3T3, mouse mastocytoma, sarcoma, lymphoma and firosarcoma) were harvested with trypsin and EDTA and then centrifuged. The cells were resuspended in DMEM/BSA. The lower compartment of the invasion chamber was filled with fibroblast-conditioned medium (FCM) which was used as a chemoattractant. The lower compartment was overlaid with PET membrane precoated with Matrigel basement membrane matrix. The cells (1.6×10⁵/well) were seeded in the upper compartment of the prehydrated Matrigel coated invasion chambers in the absence or presence of reduced rhUG and incubated at 37° C. for 24 hours in a humidified incubator. The cells which invaded the Matrigel and attached to the lower surface of the filter were stained with Giemsa. The upper surface of the filter was scraped with moist cotton swabs to remove Matrigel and non-migrated cells. The chamber was washed with water, the migrated cells were counted under an inverted microscope and photomicrographs (120×) were taken by using a Zeiss photomicroscope, Axiovert 405M.

In summary, reduced rhUG mediates a response in some tumor cell types in which the invasive phenotype is converted to a non-invasive phenotype.

Binding Studies

In order to elucidate the mechanism by which uteroglobin mediates the non-invasive phenotype, specific binding of radiolabeled rhUG to thses cells was examined. The rhUG (20 μg) was radioiodinated using sodium [¹²⁵I]iodide (2 mCi; carrier free IODO-BEADS. The reaction was carried out in 150 μl PBS, pH 7.4 at 25° C. for 10 min and ¹²⁵I-rhUG was purified by Sephadex G-25 spun column chromatography (1200× g for 4 min). The specific activity of purified carrier-free ¹²⁵I-rhUG was 25 μCi/μg. The confluent cells (NIH 3T3, mouse mastocytoma, sarcoma, lymphoma and fibrosarcoma), in 12-well plates, were washed once with PBS, pH 7.4 and then incubated with varying concentrations of reduced ¹²⁵I-UG in 1 ml of Hank's balanced salt solution (HBSS), pH 7.6, containing 0.5% BSA in the absence or presence of excess unlabeled reduced hUG at room temperature for 2 h. The UG was reduced in the presence of 10 mM DTT at 37° C. for 15 min. The reaction was stopped by rapid removal of unbound ¹²⁵I-UG and the cells were washed three times with PBS, pH 7.4 and solubilized in 1 N NaOH followed by addition of equal volume of 1N HCl. The radioactivity was measured by gamma counter (ICN Biomedicals, model 10/600 plus) with a counting efficiency of approximately 80%. The specific binding was calculated by subtracting the nonspecific binding from the total binding. The binding data were analyzed by scatchard plot using LIGAND computer program and the results are shown in FIG. 8.

The Scatchard analysis of steady state binding of ¹²⁵I-rhUG (reduced) indicates the presence of a single class of specific binding with a dissociation constant (Kd) of 20 nM using NIH3T3 cells. The dissociation constants for ¹²⁵I-rhUG (reduced) binding to mastocytoma, sarcoma, and lymphoma were comparable with values betweeen 20-25 nM. Non-reduced homodimeric ¹²⁵I-rhUG was also tested for binding to these cells and yielded Kd's between 30-35 nM for the mastocytoma, sarcoma, and lymphoma cell types. No binding of either reduced or non-reduced ¹²⁵I-rhUG was detected using the fibrosarcoma cells.

Affinity Crosslinking Experiments

To further chracterize the UG-binding sites on these cells, affinity crosslinking studies were performed with disuccinimidyl suberate (DSS) using ¹²⁵I-rhUG (reduced) in the absence and presence of unlabeled, reduced rhUG. The DSS crosslinking agent covalently couples protein molecules that are in very close contact with each other. When the unlabeled protein is added, it competes for the binding sites with the labeled protein, demonstrating the binding specificity for uteroglobin only.

Confluent cells (NIH 3T3, mouse mastocytoma, sarcoma, lymphoma and fibrosarcoma) grown in six-well plates, were washed with PBS, pH 7.4 and incubated with reduced ¹²⁵I-UG (3.0 nM) in 2.0 ml of HBSS, pH 7.6 containing 0.1% BSA in the absence or presence of unlabeled reduced UG (1 μM) for 2 h at room temperature. After washing with PBS, the cells were incubated further with 0.20 mM DSS in 2.0 ml. HBSS, pH 7.6 for 20 min. The reaction was terminated by adding 50 mM Tris-HCl buffer, pH 7.5, and cells were scraped, collected by centrifugation at 10,000× g for 15 min, and lysed in 60 μl of 1% Triton X-100 solution containing 1 mM PMSF, 20 μg/ml leupeptin and 20 mM EDTA. The supernatants (30 μl) obtained by centrifugation at 10,000× G for 15 min were suspended in sample buffer in the presence of 5% β-mercaptoethanol, boiled for 5 min and electrophoresed on 4-20% gradient sodium dodecyl sulfate (SDS)-polyacrylamide gel (Bio-Rad). The gels were briefly stained with Coomassie blue, dried in a Bio-Rad gel dryer, and autoradiographed using Kodak X-Omat Ar x-ray film.

The results of affinity crosslinking of hUG-binding proteins on NIH 3T3 (lanes 1-3), mastocytoma (lanes 4-5), sarcoma (lanes 6-7) and lymphoma (lanes 8-9) cells are shown in FIG. 9 (lane 1:(−) DSS; lane 2:(+) DSS; lane 3: (+) unlabeled hUG, (+) DSS; lane 4: (+) DSS; lane 5: (+) unlabeled hUG, (+) DSS; lane 6: (+) DSS; lane 7: (+) unlabeled reduced hUG, (+) DSS; lane 8: (+) DSS and lane 9: (+) unlabeled reduced hUG, (+) DSS). Note the presence of a 49 kDa protein band, in addition to the 190 kDa band and the decreased intensity of both bands when non-radioactive but reduced hUG was added to the reaction mixture for competition.

In summary, rhUG (reduced) mediates the loss of the invasive phenotype in certain tumor cell lines. The tumor cells that are susceptible to this rhUG-mediated behavioral change possess a single class of specific rhUG-binding activity with a low dissociation constant of approximately 20-30 nM. This rhUG binding activity is associated with specific proteins with molecular masses close to 190 kDa and 49 kDa. The invasiveness of fibrosarcoma cells, which lack the rhUG binding activity, is not affected by the presence of rhUG. Taken together, these data indicate that exogenous rhUG (either reduced or non-reduced) mediates a loss of invasiveness in tumor cells possessing the uteroglobin receptor(s).

### Example 15

### Purification of Uteroglobin Receptor(s) by Uteroglobin Affinity Chromatography

In order to purify the uteroglobin receptor(s), the tissue distribution of the receptor was analyzed using the ¹²⁵I-rhUG binding assay in several bovine tissues. During this process, the UG-binding activity was found to be primarily found in the membrane fractions of tissues and cells, indicating that the uteroglobin receptor(s) is located in the cell membrane.

Membranes were prepared from bovine heart, spleen, trachea, lung, liver and aorta. Bovine spleen was found to be enriched in UG and was chosen for further purification. The bovine spleens were homogenized in 10 mM NaHCO₃ buffer, pH 8.0. The homogenate was centrifuged at 600× g for 10 min at 4° C. The supernatant was centrifuged at 24,000× g for 60 min. The pellets were solubilized with 50 mM Tris-HCl buffer, pH 7.4, containing 1% Triton X-100, 10 μg/ml leupeptin, 2mMEDTA, and 0.4 mM PMSF by stirring at 4° C. for 6 h. The supernatant was collected by centrifugation at 24,000× g for 90 min and applied to CNBr-activated Sepharose 4B-coupled UG affinity column. The Sepharose 4B-coupled UG affinity column was prepared according to the instruction of the manufacturer (Pharmacia). The UG-receptor protein was eluted from the column using 0.1 M glycine-HCl,-pH 3.0 containing 0. 1% Triton X-100, 10 μg/ml leupeptin, 2 mM EDTA and 0.4 mM PMSF and neutralized immediately with 2M Tris-HCl, pH 8.0 The fraction containing the UG-binding proteins was detected by ¹²⁵I-UG binding and affinity crosslinking assay. The homogeneity of the purified receptor was checked by SDS-PAGE followed by silver staining (BIO-RAD).

The results are shown in FIG. 10. Note the presence of two protein bands with apparent molecular masses of 180 and 40 kDa, respectively, that are clearly visible. In addition, a third faint band with an apparent molecular mass of 32 kDa is also detectable. Thus, the uteroglobin receptor(s) that mediates the suppression of tumor cell invasiveness in vitro has been purified by uteroglobin affinity chromatography.

### Example 16

### Regulation of Expression of Uteroglobin Receptors by Cytokines

The expression of the uteroglobin receptors in response to several mediators of inflammation was investigated in NIH 3T3 cells in order to better understand the potential role of the receptor in inflammation and immunomodulation. Previous reports indicated the human uteroglobin mediated the response of human macrophages and lymphocytes to certain cytokines (Deirynck et al., 1995; 1996), suggesting a role for uteroglobin as an immunosuppressor. The effect of hUG was to suppress the IL-2 mediated transcriptional activation and de novo synthesis of IFN-γ and TNF-α. Such alterations in intracellular regulatory processes result from a signal transduction pathway in which extracellular hUG and its receptor must participate. Therefore, there is a possibility of effecting beneficial changes in the cytokine network during the immune and inflammatory responses, by manipulating the UG receptor signal transduction pathway.

The NIH 3T3 cells were cultured as described and the immune mediateors were added with and without rhUG. The levels of the UG receptor(s) were determined by binding of ¹²⁵I-rhUG followed by affinity cross-linking and SDS-Page analysis. The results are shown in FIG. 11. A considerable enhancement in intensity of the radioactive bands representing the UG-binding protein(s) followed treatment of the cells with LPS and IL-6, respectively, compared with the control. However, this difference is not apparent when the cells are treated with PMA, PDGF, TNF∝ and IFN-γ. These results provide preliminary evidence that the UG receptor is responsive to the presence of cytokines (IL-6) and pro-inflammatory mediators (LPS).

### Example 17

### Demonstration of Autocrine and Paracrine Loops in UG-Suppressible Tumor Cell Lines

In order to determine the possible role(s) of UG in suppressing the invasion of the extracellular matrix (ECM) by cancer cells, four human cell lines were studied, each of which is derived from the adenocarcinomas of the uterus and the prostate. These cell lines were chosen because the normal epithelia in these organs constitutively express the UG gene at a relatively high level. Initially, it was desirable to determine whether the adenocarcinoma-derived cell lines express UG-mRNA and UG-protein by using RT-PCR and immunoprecipitation followed by Western blotting, respectively.

Human UG Eukaryotic Expression-vector Construction, Cell Culture, Transfection and G418 Resistant Clone Selection:

The hUG cDNA cloned in pGEM 4Z (G. Mantile, L. Miele, E. Cordella-Miele, A.B. Mukherjee, J. Biol. Chem. 268, 20343 (1993)) was digested with EcoRI. A full length hUG-cDNA fragment was excised and subcloned into the TA vector (Invitrogen) at the EcoRI site. The orientation of the hUG-cDNA fragment was verified by DNA sequencing. This fragment was excised from the TA vector by digestion with HindIII and Xbal and then ligated into the pRC/RSV expression vector (Invitrogen) which had been predigested with HindIII and Xbal and purified by agarose gel electrophoresis.

The human lung adenocarcinoma cell line (HTB-174) was cultured in RPMI medium supplemented with 5% heat-inactivated fetal bovine serum at 37° C. with 5% CO₂ while the rest of the human tumor cell lines derived from adenocarcinomas of the uterus (HEC-lA) and prostate (HTB-81) were maintained in McCoy's SA medium supplemented with 10% FBS at 37° C. with 5% CO₂. The tumor cell lines were transfected with pRC/RSV-hUG construct or pRC/RSV plasmid as a control by electroporation. After 24 hours, G418 was added into the medium at a final concentration of 400 μg/ml. Individual G418 resistant clones were isolated and maintained in the medium with 200 μg/ml of G418 for further testing.

Detection of UG-mRNA by RT-PCR:

Total RNAs were isolated from different cell lines using RNAzol method (TEL-TEST, Inc.). The primers used in this study were described in Peri et al. 1993. Briefly, reverse transcription was carried out by using hUG-cDNA-specific primers, hUGr (5′T A C A C A G T G A G C T T T G G G C-3′). The RT-PCR product was then used for further amplification using primer hUGI (5′A T G A A A C T C G C T G T C A C C C-3′) and the primer hUGr. The PCR product was then blotted and detected by hybridization with a hUG-specific oligonucleotide probe hUGp (5′-T G A A G A A G C T G G T G G A C A C C-3′). The primers and the probe used for GAPDH mRNA detection are as follows:

| | |
|---|---|
| hGAPDH-r: | (5′-C A A A G T T G T C A T G G A T G A C C-3′, |
| hGAPDH-I: | (5′C C A T G G A G A A G G C T G G G G-3′) and |
| hGAPDH-p: | (5′-T C C T G C A C C A C C A A C T G C T T-3′). |

Immunoprecipitation and Western Blot Analysis:

The UG cDNA-transfected human endometrial adenocarcinoma (HEC-1A) and prostate carcinoma (HTB-81) cell lysates were immunoprecipitated according to the methods described in G. Mantile et al. J. Biol. Chem. 268, 20343 (1993)] Briefly, the cells were washed, lysed in lysis buffer and centrifuged. The supernatant was incubated with rabbit hUG-antibody for 1 hr and then incubated with protein A-agarose at 4° C. overnight. The bound complexes were collected by centrifugation, washed and eluted by boiling in SDS-sample buffer. Samples were electrophoresed on SDS-polyacrylamide gel and then electroblotted to the nitrocellulose membrane. For Western blot analysis, the membranes containing UG were blocked in blocking solution, washed and incubated with goat anti-UG antibody (1:250 dilution). The membranes were washed, incubated further with rabbit anti goat HRP-conjugated 1 gG (1:2000 dilution) and detected by enhanced chemiluminescence (ECL) method according to the instructions of the manufacturer (Amersham).

RT-PCR analysis of total RNA extracted from pRC/RSV-hUG-transfected and wild type adenocarcinomas of the uterus (HEC-1A) and prostate (HTB-81). The PCR products were blotted and detected by hybridization with a hUG-specific oligonucleotide probe. Amplification of the human GADPH gene was used as an internal control for RNA quality and to rule out pipeting error.

The results revealed that these cells neither express detectable levels of UG-mRNA nor UG-protein (FIG. 12a & 12b). These cells were then stably transfected with a human UG (hUG)-cDNA construct, pRC-RSV-hUG. The non-transfected and mock (vector only)-transfected cells served as controls. The results show that while UG-mRNA and UG-protein are undetectable in the control cells, UG-cDNA transfected cells express both UG-mRNA and UG-protein at a high level, validating the present system.

### Example 18

### Tumor Cell Phenotypes

To determine whether forced UG-expression in these adenocarcinoma cell lines had any effect on anchorage-independent growth and their ability to invade the ECM, two of the most important characteristics of cancer cells, they were tested for growth on soft agar and for Matrigel (ECM) invasion, respectively. As shown in FIG. 13, forced-expression of UG caused dramatic inhibition of anchorage-independent growth on soft agar (FIG. 13a) and ECM-invasion (FIG. 13b) by HEC-1A cells, transfected with pRC-RSV-hUG, but not by control or mock-transfected cells. The suppression of ECM-invasion due to pRC/RSV/hUG-transfection of HEC-1A tumor cells was about 79% compared to that of the non-transfected controls. The other cell lines derived from the adenocarcinomas of the lung, mammary gland and the prostate did not show any suppression of anchorage-independent growth on soft agar or ECM-invasion (data not shown).

To test whether treatment of the adenocarcinoma cell lines with purified hUG could suppress anchorage-independent growth on soft agar and ECM-invasion, the same assays were performed using all four cell lines mentioned above that were either non-transfected or mock-transfected. The results show that pretreatment of these cells with purified recombinant hUG dramatically suppresses anchorage-independent growth on soft agar as well as ECM-invasion by untransfected HEC-1A cells. This percentage of inhibition of ECM-invasion is very similar to that obtained when pRC-RSV-hUG-transfected HEC-1A cells were tested (FIG. 14). The above results led to further investigation of the mechanism(s) of hUG mediated suppression of anchorage-independent growth and ECM-invasion by pRC-RSV-hUG-transfected HEC-1A cells.

Soft agar assay was performed on both non-transfected and pRC/RSV-UG construct or pRC/RSV plasmid transfected uterus (HEC-1A), prostate (HTB-81) and lung (HTB-174) tumor cell lines. Cells were trypsinized and seeded on a 60 mm dish in 2 ml 0.3% noble agar containing the same culturing medium over a 5 ml basal layer of 0.5% agar containing the same medium as the seed layers. The top agar/medium contained 200 μg/ml G418 was used for pRC/RSV-UG construct or pRC/RSV plasmid transfected cells. Plates were incubated at 37° C. and 5% CO₂ for 12-14 days. The colonies were stained with medium Red stain and counted manually.

The in vitro Matrigel-invasion assay was carried out as described in Kundu et al., 1996. Briefly, when the cells reached about 80% confluence, they were trypsinized and washed twice with PBS containing 0.1% BSA. The cells were resuspended in DMEM containing 0.1% BSA and placed in the upper compartment of the Matrigel invasion chamber. The lower compartment of the chamber was filled with fibroblast conditioned medium (FCM), a chemoattractant for cell invasion, which was prepared from the supernatant of proliferating cultures of NIH 3T3 fibroblasts after incubating for 24 hours. After incubating at 37° C. for 36 hours, the cells were stained with Giemsa for 3 min. and immediately washed with absolute ethanol twice, 5 min. each. The non-invaded cells and Matrigel were scraped from the upper surface of the filter with moist cotton swabs and the chamber was washed three times with water. The invaded cells remained on the filter were counted under an inverted microscope and the percentage of the cell invasion was calculated by comparison of the invaded cells from the transfected cells with those from the control cells.

Morphology of the control cells (pRC/RSV vector alone transfected adenocarcinomas of the uterus) on soft agar was shown in FIG. 14 (a) HEC-LA, while morphology of the hUG expression construct transfected cells on soft agar was shown in FIG. 14 (b) HEC-lA/UG. The small size of the colonies in the presence of rhUG shows that uteroglobin not only suppresses human tumor cell invasiveness but tumor cell growth as well.

### Example 19

### Demonstration of UG-Receptor Binding

¹²⁵I-hUG-binding and affinity-crosslinking assays were performed to determine whether hUG exerts this effect via its receptor-mediated pathway.

¹²⁵I-UG-Binding Assay:

The radioiodination of UG and binding experiments were performed as described Kundu et al., 1996. Briefly, the UG (20 μg) was radioiodinated using ¹²⁵I-sodium iodide (2 mCi; carrier-free) and IODOBEADS. The ¹²⁵I-UG was purified by Sephadex G-25 spun column chromatography (1200× g for 4 min). The specific activity of purified ¹²⁵I-UG was 20 μCi/μg. Both the non-transfected and the human pRSV/hUG-transfected confluent HEC-LA cells in 12-well plates were washed with PBS, pH 7.4 and incubated with reduced ¹²⁵1-UG (1.5 nM) in 1 ml of Hanks Balanced Salt Solution (HBSS), pH 7.6 containing 0.1% BSA in the absence or presence of increasing concentrations (1 pM to 1 μM) of unlabeled reduced recombinant hUG at room temperature for 2 h. The cells were washed with PBS, pH 7.6 and solubilized in 1N NaOH followed by addition of equal volume of 1N HCl. The radioactivity was measured by a gamma counter. The specific binding was calculated by subtracting the non-specific binding from the total binding. Scatchard analyses of the data were performed by using LIGAND computer program. These results are identical to those shown in FIG. 8, indicating that this is the same UG receptor previously characterized.

Affinity CrossLinking Experiments:

The non-transfected and pRC/RSV/hUG-transfected adenocarcinoma cells were grown to confluence in 6-well plates. They were washed with PBS, pH 7.6 and incubated with reduced recombinant human ¹²⁵I-UG (3nM) in 2.0 ml HBSS, pH 7.6 containing 0.1% BSA in the absence and presence of unlabeled reduced hUG (250 nM) at room temperature for 2 h. Following incubation, the cells were washed and incubated further with 0.2 mM disuccimidylsuberate (DSS) in 2. ml HBSS, pH 7.6 for 20 min. The cells were scraped, collected by centrifugation (10,000× g) for 15 min and lysed in 40:1 ratio of lysis buffer (1% Triton X-100 containing 1 mM PMSF, leupeptin (20 μg/ml) and 2 mM EDTA. The supernatants were resuspended in sample buffer containing 5% B-mercaptoethanol. The samples were resolved by SDS-PAGE and autoradiographed.

The results of the present experiments show that ¹²⁵I-hUG bound only to HEC-1A cells with high-affinity (Kd=25nM) and specificity (FIG. 15a & 15b) while all other adenocarcinoma cell lines lacked this binding (data not shown). The UG-receptor was identified on HEC-1A (responder) cells but not on HTB-81 (non-responder) cells. Affinity crosslinking of ¹²⁵I-hUG with its binding proteins on non-transfected (a) and pRSV/hUG-transfected HEC-1A and HTB-81 cells, respectively. The cells were incubated with reduced ¹²⁵I-hUG in the absence and presence of unlabeled reduced hUG for binding and then crosslinked with DSS (lane 1: (−)DSS; lane 2: (+)DSS and lane 3: (+)hUG +DSS). The results of affinity-crosslinking experiments using ¹²⁵I-hUG demonstrate the presence of both 190 kDa and 49 kDa UG binding-proteins in HEC-lA cells (FIG. 15b) but not on other three adenocarcinoma cell lines tested (not shown). Thus, forced UG-expression or treatment of the cells with purified hUG suppress ECM-invasion of only those cells that express the hUG-binding proteins.

### Example 20

### Tumorigenesis in UG-deficient Mice

Thusfar, a total of 16 UG −/− mice, (with late onset disease), surviving for over one year from birth, exhibit not only renal impairment, but also develop tumors. That is, all 16 mice (100%) of the aged UG-deficient mice, to date, have developed tumors. These tumors originate in a variety of tissues and represent a variety of tumor types still being characterized. The results nonetheless demonstrate the significance of UG deficiency in the development of cancer and indicate the potential utility for rhUG in the treatment and/or prevention of cancer.

### Example 21

### Identification of UG as an HCG-Associated Factor (HAF)

A recent report describes an HCG-(human chorionic gonadotropin) associated factor, termed HAF, found in the urine of women during early pregnancy, that (1) blocks tumorigenesis and metastasis of Karposi's sarcoma; (2) blocks HIV infection; and (3) stimulates hematopoiesis (Lunardi-Iskandar et al, 1995; 1998). HAF co-purifies with HCG from human urine and may form a complex with HCG. Human UG is elevated in the urine of women during early pregnancy. Both UG and HAF are low molecular weight proteins (15-30 kDa) and both suppress tumor cell invasiveness. Preliminary in vitro studies show that ¹²⁵I-rhUG and HCG (obtained from Ayerst Labs, Inc.) do in fact, form a tightly bound complex, which suggests that uteroglobin and HAF are the same protein.

While the invention has been described in connection with what is presently considered to be the most practical and preferred embodiments, it is to be understood that the invention is not to be limited to the disclosed embodiments but, on the contrary, is intended to cover various modifications and equivalent arrangements included within the spirit and scope of the appended claims.

### REFERENCES

1. Levin, S. W. et al., Life Sci. 38: 1813-1819 (1986);

2. Singh G. et al., Biochem. Biophys. Acta. 1039: 348-355(1990);

3. Mantile, G. et al., J. Biol. Chem 268: 20343-20351 (1993);

4. Singh, G. et al., J. Histochem. Cytochem. 36: 73-80 (1987);

5. Bernard, A. et al., Clin. Chem. 38: 434-435 (1992);

6. Dhanireddy, R. et al., Pediatric Res. 23: 463A (1988);

7. Dhanireddy, R. et al., Pediatric Res. 33: 323A (1993);

8. Piomelli, D., Op. In Cell Biol. 5: 274-280(1993);

9. Krishnan, R. S. et al., Science 158: 490-492 (1967);

10. Beier, H. Verhandl Deut. Zool. Ges. Heidelberg (1968);

11. Umland, T. C. et al., Nature Stuct. Biol. 1: 538-545 (1994);

12. Hard, T. et al., Nature. Struct. Biol. 2: 938-989 (1995);

13. Umland, T. C. et al., Nature Struct. Biol. 2: 919-922(1995);

14. Stripp, B. R. et al., Am. T. Physio. 271 (Lung Cell. Mol. Physiol. 15): L656-L664 (1996);

15. Lesur, O. et al., Am. T. Respir. Crit. Care Med. 152: 290-297 (1995);

16. Glaser, K. B., Adv. Pharmacol. 32: 31-66 (1995);

17. Tykka, H. T. et al., Scand. J. Gastroenterol. 20: 5-12 (1985);

18. Sheuer, W., Klin. Wochenschr. 67: 153-159 (1989);

19. Barnes, H. J. et al., J. Mol. Biol., Feb. 23, 1996;

20. Aoki, A. et al., Mol. Hum. Reprod. 2: 419-497 (1996);

21. Anderson and Kurkland, Microbiological Reviews 54: 198-210 (1990);

22. Miele, L. et al., J. Biol. Chem. 265: 6427-6435 (1990);

23. Coalson, J. J. et al., Exp. Mol. Pathol. 37: 355-360 (1982);

24. Nagy, A. et al., Proc. Natl. Acad. Sci. 90: 8424 (1993);

25. Capecchi, M. R., Science, 244: 1288 (1989);

26. Harlow, E. and Lane D. Antibodies: A Laboratory Manuel, 1st Ed. Cold Spring Harbor Laboratory Press, Cold Spring Harbor, N.Y., 1988;

27. Mantile, G. et al., J. Biol. Chem. 267: 20343 (1993);

28. Ruoslahti, E. Ann Rev. Biochem. 57: 375 (1988);

29. R. O. Hynes, Fibronectins, New York: Springer-Verlag (1990);

30. Chernousor, M. A. et al., J. Biol. Chem. 266: 10857 (1991);

31. Zhang, Q. et al., J. Cell. Biol. 127: 1447 (1994);

32. Wu, C. et al. Cell 83: 715 (1995);

33. Zhang, Q. et al., J. Biol. Chem. 271: 33284 (1996);

34. Border, W. A. et al., J. Clin. Invest. 90: 1 (1992);

35. Peri, A., et al., J. Clin. Invest. 92: 2099 (1993);

36. Peri, A. et al., J. Clin. Invest. 96: 343 (1995);

37. Oh, E. et al., Proc. Natl. Acad. Sci. (USA) 78: 3218 (1981);

38. Mosher, D. F. et al., Curr, Biol. 4: 810 (1992).

39. R. S. Krishnan, J.C. Daniel Jr., Science 158, 490 (1967).

40 H. M. Beier, Biochim Biophys Acta 160, 28 (1968).

41. A. Peri, E. Cordell-Miele, L. Miele, A.B. Mukherjee, J. Clin Invest 92, 2099 (1993).

42. G. Singh et al. Biochim. Biophys. Acta 950, 329 (1988).

43. J. Jackson, R. Turner, J. N. Keen, R. A. Brooksbank and E. H. Cooper, J. Chromatogr. 452, 359 (1989).

44. M. J. Beato, Steroid Biochem. 7,327 (1976); M. Gillener et al., J. Steroid Biochem. 31,27 (1988).

45. K. Diaz Gonzalez and A. Nieto, FEBS Lett. 361, 255 (1995).

46. M. A. Watson and T. P. Fleming, Cancer Res. 56,860 (1996)

47. M. A. Watson, C. Darrow, D. B. Zimonjic, N. C. Popescu, T. P. Fleming, Oncogene 16 (2), 817 (1998).

48. L. Miele, E. Cordella-Miele, A.B. Mukherjee Endocrine Reviews, 8, 474 (1987).

49. L. Miele, E. Cordella-Miele, G. Mantile, A. Peri, A. B. Mukherjee J. Endocrinol. Invest., 17,679 (1994).

50. L. Miele, E. Cordella-Miele, A. Facchiano, A. B. Mukherjee, Nature 335, 726 (1988).

51. L. Miele, E. Cordella-Miele, J Biol Chem 265,6427 (1990).

52. G. Camussi, C. Tetta, F. Bussolino, C. Baglioni, J.Exp.Med. 171,913 (1990).

53. S. Lloret, J. J. Moreno, Biochem. Pharm. 50 (3), 347 (1995).

54. G. Mantile, L. Miele, E. Cordella-Miele, G. Singh, S. L. Katyal, A. B. Mukherjee, J Biol Chem 268, 20343 (1993);

55. G. Vasanthakumar, R. Manjunath, A.B. Mukherjee, H. Warabi, E. Schiffman, Biochem, Pharmacol. 37(3), 389 (1988).

56. R. Manjunath, R. et al. Biochem. Pharmacol. 36 (5), 741 (1987).

57. J. G. Vostal, A. B. Mukherjee, L. Miele, N. R. Shulman, Biochem. Biophys. Res. Commun. 165(1), 27 (1989).

58. A. Melchiori et al. Anticancer Res. 10(1), 37 (1990).

59. G. C. Kundu, G. Mantile, E. Cordella-Miele, A. B. Mukherjee, Proc. Natl. Acad. Sci. USA. 93, 2915 (1996).

60. K. Diaz Gonzalez, A. Nieto, FEBS Lett. 361, 255 (1995).

61. Z. Zhang et al. DNA Cell Biol. 16 (1), 73 (1997).

62. B. C. Misra, E. S. Srivatan, Am J. Hum. Genet. 455, 65 (1989).

63. G. A. Lammie et al. Oncogene 6, 439 (1991).

64. G. A. Lammie, G. Peters, In Cancer Cells Vol. 3 (11), 413 (1991), Cold Spring Harbor Laboratory Press, Cold Spring Harbor, N.Y.

65. S. Brookes, et al. Genes Chromosomes & Cancer 4, 290 (1992).

66. R. A. Jesudasan, et al., Anticancer Res. 14, L1727 (1994).

67. G. M. Hampton, et al. Proc. Natl. Acad. Sci. USA. 91,6953 (1994).

68. R. A. Jesudasan, et al. Am. J. Hum. Genet. 56, 705 (1995).

69 P. J. Saxon, E. S. Srivatan, E. J. Stanbridge, EMBO J. 5, 3461 (1986).

70. M. Koi, et al., Mol. Carcinogenesis 2, 12 (1989).

71. I. Linnoila et al., Am. J. Clin. Path. 97, 235 (1992).

72. J. L. Broers et al. Lab. Invest. 66, 337 (1992).

73. A. Sandmoller et al., Cell Growth Differ. 6, 97 (1995).

74. F. J. DeMayo et al., Am. J. Physiol. 261, L70 (1991).

75. A. Weerartna et al. Clin. Cancer Res. 3, 2295 (1997).

76. A. B. Mukhejee, L. Murty, J. Y. Chou, Mol. Cell. Endocrinol. 94, R15 (1993).

What is claimed is:

1. A method of preventing or treating primary cancer cell growth comprising administering to a patient in need of such prevention or treatment a tumor-suppressive effective amount of recombinant human uteroglobin (rhUG) or a fragment or derivative thereof.

2. The method of claim 1 further comprising targeting a uteroglobin receptor by administering said tumor-suppressive effective amount of rhUG.

3. A pharmaceutical composition comprising a tumor-suppressive effective amount of rhUG and a pharmaceutically acceptable carrier or diluent.

4. The pharmaceutical composition of claim 3 wherein said rhUG has a purity of about 75% to about 100%.

5. The pharmaceutical composition of claim 3 wherein said rhUG has a purity of about 90% to about 100%.

6. The pharmaceutical composition of claim 3 wherein said rhUG has a purity of at least 95%.

7. The pharmaceutical composition of claim 3 wherein said rhUG is reduced and monomeric.

8. A method of preventing or treating tumor metastasis by inhibiting fibronectin aggregation and/or deposition comprising administering to a patient in need of such prevention or treatment a fibronectin inhibiting effective amount of rhUG or a fragment or derivative thereof.

9. The method of claim 8 further comprising targeting a uteroglobin receptor by administering said fibronectin inhibiting effective amount of rhUG.

10. A method of stimulating hematopoiesis comprising administering to a patient in need of such stimulation a hematopoiesis stimulating effective amount of rhUG or a fragment or derivative thereof.

11. The method of claim 10 further comprising targeting a uteroglobin receptor by administering a hematopoiesis stimulating effective amount of rhUG.

12. A pharmaceutical composition comprising a hematopoiesis stimulating effective amount of rhUG or a fragment or derivative thereof and a pharmaceutically acceptable carrier or diluent.

13. The pharmaceutical composition of claim 12 wherein said rhUG has a purity of about 75% to about 100%.

14. The pharmaceutical composition of claim 12 wherein said rhUG has a purity of about 90% to about 100%.

15. The pharmaceutical composition of claim 12 wherein said rhUG has a purity of at least 95%.

16. A method of screening a sample comprising one or more compounds, peptides and/or proteins for uteroglobin structural analogs and/or UG-receptor ligands comprising
(a) contacting said sample with a purified uteroglobin receptor(s); and
(b) detecting a binding interaction between said receptor(s) and said sample, which binding interaction is indicative of the presence of a uteroglobin structural analog and/or UG-receptor ligand in said sample.

17. A kit for screening of uteroglobin structural analogs and/or UG-receptor ligands comprising purified uteroglobin receptor(s).

18. A method of purifying a uteroglobin receptor(s) from a sample comprising
(a) contacting said sample with rhUG bound to a solid support; and
(b) eluting a purified sample of uteroglobin receptor(s) from said solid support.

19. A method of preparing reduced rhUG comprising contacting oxidized rhUG with a reducing agent for a time and temperature sufficient to reduce rhUG.

20. The method of claim 19 wherein said reduced rhUG is monomeric.

21. A method of generating antibodies to a uteroglobin receptor(s) comprising immunizing an animal with a purified uteroglobin receptor(s) and isolating antibodies to said receptor(s).
